(19) United States
(12) Patent Application Publication
(10) Pub. No.: US 2018/0338705 A1
(43) Pub. Date: Nov. 29, 2018

(54) MOBILE TERMINAL APPARATUS, PROGRAM, AND BIOLOGICAL INFORMATION MEASUREMENT SYSTEM

(71) Applicant: KYOCERA Corporation, Kyoto-shi (JP)

(72) Inventors: Tomoaki KONISHI, Yokohama-shi (JP); Hiromi AJIMA, Kawasaki-shi (JP)

(21) Appl. No.: 15/990,691

(22) Filed: May 28, 2018

(30) Foreign Application Priority Data
May 29, 2017 (JP) 2017-105837

(51) Int. Cl. A61B 5/08; A61B 5/00; A61B 5/11; A61B 7/00

(52) U.S. Cl. CPC A61B 5/0816; A61B 5/0002; A61B 7/003; A61B 5/4812; A61B 5/11

(57) ABSTRACT

A mobile terminal apparatus capable of reducing inconvenience, a program, and a biological information measurement system are provided. A mobile terminal apparatus includes a gyro sensor configured to detect a motion factor and a controller configured to perform measurement operation of biological information of a user on the basis of the motion factor detected due to a movement of the user. The controller starts or stops the measurement processing on the basis of the motion factor.

### CROSS-REFERENCE TO RELATED APPLICATION

The present application claims priority to and the benefit of Japanese Patent Application No. 2017-105837 filed May 29, 2017, the entire contents of which are incorporated herein by reference.

### TECHNICAL FIELD

The present disclosure relates to a mobile terminal apparatus, a program, and a biological information measurement system.

### BACKGROUND

Mobile phones which are configured to perform predetermined output on the basis of information acquired from a user are conventionally known. For example, a mobile phone which is configured to detect the respiratory sound of a user and, when apnea is detected, output an alarm is known.

### SUMMARY

According to an embodiment, a mobile terminal apparatus includes a gyro sensor configured to detect a motion factor and a controller configured to execute measurement processing of biological information on the basis of the motion factor detected due to a movement of a user. The controller starts or stops the measurement processing on the basis of the motion factor.

According to an embodiment, a program causes a mobile terminal apparatus to execute a step of detecting a motion factor by using a gyro sensor and a step of starting or stopping measurement processing of biological information of a user on the basis of the motion factor detected due to a movement of the user.

According to an embodiment, a biological information measurement system includes a mobile terminal apparatus equipped with a gyro sensor configured to detect a motion factor. The biological information measurement system includes an external apparatus that includes a controller configured to start or stop, on the basis of the motion factor detected due to a movement of the user, measurement processing of the biological information of the user based on the motion factor.

### BRIEF DESCRIPTION OF THE DRAWINGS

In the accompanying drawings:

FIG. 1 is a functional block diagram schematically illustrating a configuration of a mobile terminal apparatus according to an embodiment;

FIGS. 2A and 2B are perspective views schematically illustrating an exterior of the mobile terminal apparatus according to the embodiment;

FIG. 3 is a diagram schematically illustrating thee aortas in a human body;

FIGS. 4A and 4B are diagrams illustrating examples of an abutment state of a measured part and an abutment;

FIGS. 5A, 5B, and 5C are diagrams illustrating usage modes of the mobile terminal apparatus according to an embodiment;

FIGS. 6A and 6B are diagrams illustrating usage modes of the mobile terminal apparatus according to an embodiment;

FIG. 7 is a graph illustrating an example of a pulse wave acquired by a sensor;

FIG. 8 is a graph illustrating a temporal variation in a calculated AI;

FIG. 9 is a graph illustrating the calculated AI and a result of measurement of a blood glucose level;

FIG. 10 is a graph illustrating a relation between the calculated AI and the blood glucose level;

FIG. 11 is a graph illustrating the calculated AI and a result of measurement of a neutral lipid level;

FIG. 12 is a flowchart illustrating a process for estimating blood fluidity and states of glucose metabolism and lipid metabolism;

FIG. 13 is a graph illustrating an example of a waveform of respiration acquired by a sensor;

FIG. 14 is a diagram illustrating an example of a waveform in which a pulse wave and respiration are combined;

FIG. 15 is a diagram illustrating a usage mode of the mobile terminal apparatus according to an embodiment;

FIGS. 16A and 16B are diagrams schematically illustrating examples of respiration rhythm;

FIG. 17 is a diagram schematically illustrating a motion factor acquired by the mobile terminal apparatus;

FIG. 18 is a flowchart illustrating an example of a process for starting measurement operation of the biological information performed by the mobile terminal apparatus;

FIG. 19 is a flowchart illustrating an example of a process for stopping the measurement operation of the biological information performed by the mobile terminal apparatus;

FIG. 20 is a diagram illustrating a usage mode of the mobile terminal apparatus according to an embodiment of the present disclosure; and

FIG. 21 is a diagram schematically illustrating a configuration of a biological information measurement system according to an embodiment of the present disclosure.

### DETAILED DESCRIPTION

However, the conventionally known mobile phone does not autonomously start or stop acquiring information about the user. Therefore, from the perspective of the user, operation of the mobile phone may feel inconvenient.

It would be helpful to provide a mobile terminal apparatus, a program, and a biological information measurement system which are capable of reducing this inconvenience.

Hereinafter, embodiments will be described in detail with reference to the drawings.

In an embodiment described herein, a mobile terminal apparatus is assumed to be, for example, a mobile phone such as a smartphone. However, the mobile terminal apparatus is not limited to a mobile phone such as a smartphone and may be a feature phone. The mobile terminal apparatus is not necessarily limited to a mobile phone and may be various mobile terminal apparatuses including, for example, a tablet computer, a remote controller configured to remotely control an electronic device, a digital camera, a laptop computer, etc. In short, the mobile terminal apparatus may be any mobile terminal apparatus having the functions described in the embodiments described herein.

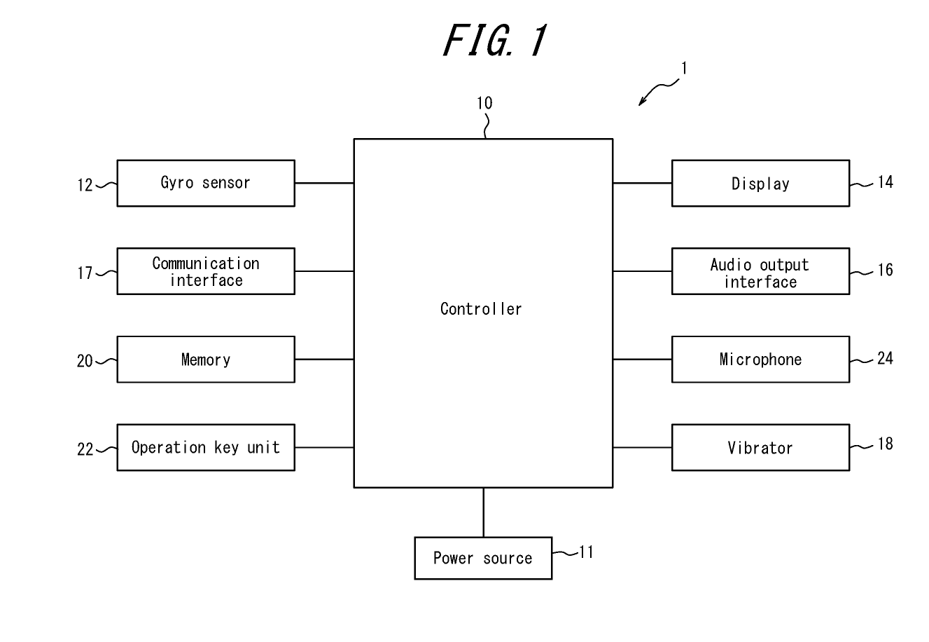

FIG. 1 is a functional block diagram schematically illustrating a configuration of the mobile terminal apparatus according to an embodiment. As illustrated in FIG. 1, the mobile terminal apparatus 1 includes a controller 10, a power source 11, a gyro sensor 12, a display 14, an audio output interface 16, a communication interface 17, a vibrator 18, and a memory 20. The mobile terminal apparatus 1 also includes an operation key unit 22 and a microphone 24.

The controller 10 includes a processor configured to control and manage the mobile terminal apparatus 1 in its entirety including each functional block thereof. The controller 10 includes a processor such as a CPU (Central Processing Unit) configured to execute a program specifying a control procedure and a program for measuring biological information of a user. Such programs are stored in a storage media such as the memory 20. The controller 10 performs control to implement various functions of the mobile terminal apparatus 1. For example, when the mobile terminal apparatus 1 is a smartphone, the controller 10 performs control to implement functions associated with telephone calls or data communications and functions associated with execution of application programs.

The power source 11 includes a battery and supplies power to each element of the mobile terminal apparatus 1. The mobile terminal apparatus 1 receives power supply from the power source 11 or an external power source during operation.

The gyro sensor 12 detects a movement of the mobile terminal apparatus 1 as a motion factor by detecting an angular velocity of the mobile terminal apparatus 1. The gyro sensor 12 is, for example, a triaxial vibration gyro sensor configured to detect an angular velocity from deformation of a structure caused by a Coriolis force acting on a vibrating arm. This structure may be made of quartz crystal, a piezoelectric material such as piezoelectric ceramics, or the like. The gyro sensor 12 may be formed with a structure formed from material such as silicon by employing MEMS (Micro Electro Mechanical Systems) technology. The gyro sensor 12 may be of another type, such as an optical gyro sensor. The controller 10 may measure an orientation of the mobile terminal apparatus 1 by performing time integration of the angular velocity acquired by the gyro sensor 12.

The gyro sensor 12 is, for example, an angular velocity sensor. However, the gyro sensor 12 is not limited to an angular velocity sensor. The gyro sensor 12 may detect an angular displacement of the mobile terminal apparatus 1 serving as a motion factor. The gyro sensor 12 detects a motion factor to be processed as a self-control factor. The motion factor detected by the gyro sensor 12 is transmitted to the controller 10.

The controller 10 acquires the motion factor from the gyro sensor 12. The motion factor includes an indicator of the displacement of the mobile terminal apparatus 1 caused by pulsation in a measurement part of the user. The controller 10 determines the pulsation of the user on the basis of the motion factor. The controller 10 measures the biological information on the basis of the pulsation of the user. Also, the controller 10 performs a starting process or a stopping process of a measurement process of the biological information. The processes performed by the controller 10 will be described in detail later.

The display 14 includes a display device such as a liquid crystal display, an inorganic EL (Electro-Luminescence) panel, or an inorganic EL (Electro-Luminescence) panel. The display 14 displays characters, images, symbols, and/or shapes. The display 14 may be configured with a touchscreen display that has a touchscreen function in addition to a display function. In this case, the touch screen detects contact by a user's finger or a stylus pen. The touchscreen may detect the positions of contacts made by a plurality of fingers or stylus pens in respect to the touchscreen. The touchscreen may employ any appropriate detection method including a capacitive method, a resistive film method, a surface acoustic wave method (or an ultrasound method), an infrared method, an electromagnetic induction method, and a load detection method. The capacitive method may detect contact and approach by the finger or the stylus pen.

The audio output interface 16 notifies the user of information by outputting a sound. The audio output interface 16 may be configured with any appropriate speaker. The audio output interface 16 may output a sound corresponding to a sound signal transmitted from the controller 10. The user may hear a voice of the other party in a telephone call from the audio output interface 16 when, for example, talking on the mobile terminal apparatus 1. In this case, the user may hear the voice of the other party by placing the audio output interface 16 against the ear. When the mobile terminal apparatus 1 is used as a speakerphone or the like, the user may hear the voice of the other party without placing the audio output interface 16 against the ear.

The communication interface 17 transmits and receives various data with an external apparatus by performing wired communication or wireless communication. The communication interface 17 may connect to and communicate with a base station and the like to realize the function of a telephone call and/or the function of data communication with the mobile terminal apparatus 1. The communication interface 17 may transmit, for example, a measurement result of the biological information measured by the mobile terminal apparatus 1 to an external apparatus. Further, the communication interface 17 may communicate with an external apparatus that stores the biological information of the user for the purpose of health management.

The vibrator 18 notifies the user of information by generating vibration. The vibrator 18 generates vibration at any part of the mobile terminal apparatus 1 to provide a tactile sensation to the user of the mobile terminal apparatus 1. The vibrator 18 may be any member that generates vibration, such as an eccentric motor, a piezoelectric element, or an linear vibrator.

The memory 20 stores various programs and data including application programs. The memory 20 may include any non-transitory storage medium such as a semiconductor storage medium or a magnetic storage medium. The memory 20 may include various types of storage media. The memory 20 may include a combination of a mobile storage medium such as a memory card, an optical disk, and a magneto-optical disk, and a storage medium reader. The memory 20 may include a storage device used as a provisional storage area such as a RAM (Random Access Memory). The memory 20 stores various information and programs for the operation of the mobile terminal apparatus 1 and functions as a working memory. For example, the memory 20 may store data detected by the gyro sensor 12 and a result of measurement of the biological information.

The operation key unit 22 is configured with at least one operation key configured to detect input operation by the user. The operation key unit 22 may be configured with any key or button, such as a push button switch or a slide switch. The mobile terminal apparatus 1 does not necessarily need to include the operation key unit 22 when the touch screen display may be used for all input operation.

The microphone 24 detects a sound and converts the sound into a sound signal. The microphone 24 may be any suitable device that is capable of detecting a sound. The microphone 24 transmits the sound signal thus converted to the controller 10. The controller 10 may transmit a received sound signal from the communication interface 17. In this way, the user may send a sound input to the microphone 24 to the other party during, for example, a telephone call on the mobile terminal apparatus 1.

The mobile terminal apparatus 1 according to embodiments of the disclosure is not limited to have the configuration illustrated in FIG. 1. The controller 10 and the gyro sensor 12 are the constituent elements essential to the mobile terminal apparatus 1 according to the present embodiment for measurement of the biological information. In the mobile terminal apparatus 1 according to the present embodiment, therefore, elements other than the essential constituent elements may be omitted, or other elements may be added, as necessary. Although the controller 10 and the gyro sensor 12 are the constituent elements essential to the mobile terminal apparatus 1 according to the present embodiment for measurement of the biological information, a mobile terminal apparatus 1 that does not measure the biological information may omit (i.e. not include) the gyro sensor 12. In this case, for example, an external member such as a case or an attachment attachable to the mobile terminal apparatus 1 may include the gyro sensor 12.

The mobile terminal apparatus 1 may measure the biological information at a measured part of the user. The measured part may be the body of the user (i.e. the user of the mobile terminal apparatus 1), as described later. The mobile terminal apparatus 1 measures the biological information of the user on the basis of movement of the body serving as the measured part.

The biological information measured by the mobile terminal apparatus 1 includes at least one of, for example, a blood component, a pulse wave, pulsation, and a pulse wave velocity. The blood component includes, for example, a glucose metabolism state and a lipid metabolism state. The glucose metabolism state includes, for example, a blood glucose level. The lipid metabolism state includes, for example, a lipid value. The lipid value includes neutral fat, total cholesterol, HDL (High Density Lipoprotein) cholesterol, and LDL (Low Density Lipoprotein) cholesterol. The mobile terminal apparatus 1 acquires, for example, the pulse wave of the user as the biological information and measures the biological information such as the blood component on the basis of the acquired pulse wave.

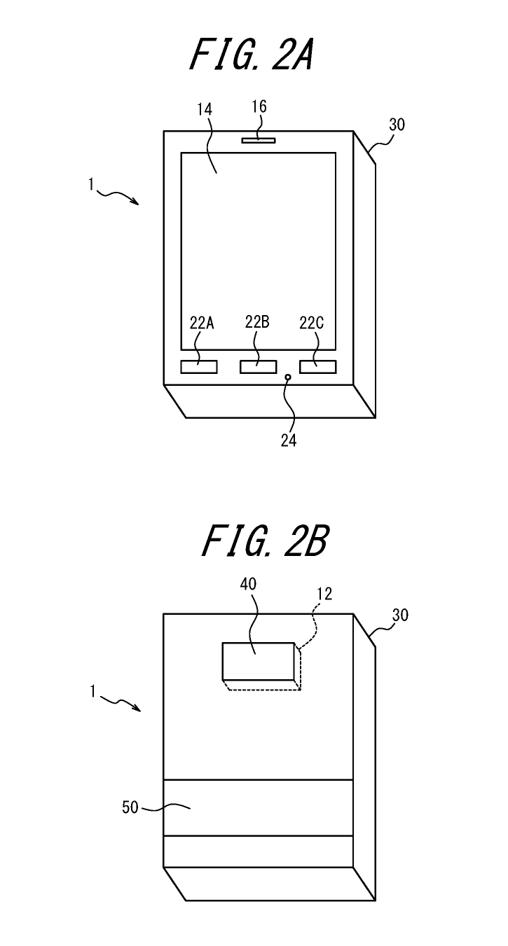

FIGS. 2A and 2B are perspective views schematically illustrating an exterior of the mobile terminal apparatus 1 according to the present embodiment. The mobile terminal apparatus 1 according to the present embodiment may be a relatively small mobile terminal apparatus such as a mobile phone, as illustrated in FIG. 1 by way of example However, the mobile terminal apparatus 1 is not limited to a mobile terminal apparatus such as a mobile phone. For example, the mobile terminal apparatus 1 may be incorporated in any mobile electronic device.

FIG. 2A is a diagram illustrating a front side of the mobile terminal apparatus 1. FIG. 2B is a diagram illustrating a rear side of the mobile terminal apparatus 1, that is, illustrating a state in which the mobile terminal apparatus 1 illustrated in FIG. 2A is turned over.

As illustrated in FIGS. 2A and 2B, the mobile terminal apparatus 1 includes a housing 30 having a substantially rectangular exterior. As illustrated in FIG. 2A, the mobile terminal apparatus 1 includes the display 14, the audio output interface 16, the operation key unit 22, and the microphone 24 on the front side. The display 14 is capable of displaying information associated with the measurement processing of the mobile terminal apparatus 1. By viewing the display on the display 14, the user may confirm a measurement state while measuring the biological information. By viewing the display on the display 14, the user may also know a result of the measurement of the biological information. By viewing the display on the display 14, further, the user may confirm whether the biological information is correctly measured. The display 14 may also display information such as time.

When the mobile terminal apparatus 1 functions as a mobile phone, the audio output interface 16 outputs the voice of the other party. When the mobile terminal apparatus 1 measures the biological information, the audio output interface 16 may inform the user of the start or end of the measurement by outputting a sound when the mobile terminal apparatus 1 starts or ends the measurement of the biological information. The audio output interface 16 may output a sound for notifying the user that the measurement is being performed. The mobile terminal apparatus 1 may output a sound to induce the user to fall asleep according to a sleep-inducing operation, which will be described later.

In the example illustrated in FIG. 2A, the operation key unit 22 includes operation keys 22A, 22B, and 22C. The number and arrangement of the keys of the operation key unit 22 is not limited to those illustrated in FIG. 2A and may be varied on the basis of a specification for the mobile terminal apparatus 1 and the like. For example, although the operation key unit 22 is arranged only on the front surface of the mobile terminal apparatus 1 in the example illustrated in FIG. 2A, the operation key unit 22 may be arranged on a side surface or the rear surface of a body of the mobile terminal apparatus 1. In the mobile terminal apparatus 1, the operation key unit 22 may be configured with a switch such as a button for starting the measurement of the biological information.

As described above, the microphone 24 detects the voice of the user mainly when the mobile terminal apparatus 1 is functioning as a mobile phone. Although one microphone 24 is arranged on the front surface of the mobile terminal apparatus 1 in the example illustrated in FIG. 2A, the number and position of the microphone 24 may be varied on the basis of a specifications for the mobile terminal apparatus 1 and the like.

As illustrated in FIG. 2B, the mobile terminal apparatus 1 includes an abutment 40 and a support 50 on the rear surface. In the example illustrated in FIG. 2B, the abutment 40 and the support 50 are substantially flush with the rear surface of the housing 30. However, at least one of the abutment 40 and the support 50 may be protrude from the rear surface of the housing 30. As illustrated in FIG. 2B, the abutment 40 and the support 50 are fixed to the mobile terminal apparatus 1 on the rear surface of the housing 30. At least one of the abutment 40 and the support 50 may be, for example, attached to the mobile terminal apparatus 1 in a non-detachable manner. At least one of the abutment 40 and the support 50 may be, for example, detachably attached to the mobile terminal apparatus 1.

On the rear surface of the housing 30, the abutment 40 and the support 50 are fixed in a manner linearly extending along a transverse direction of the rear surface. Lengths of the abutment 40 and the support 50 in the transverse direction of the rear surface of the housing 30 may be, for example, shorter than a transverse length of the housing 30. A relation between the length of the abutment 40 and the length of the support 50 in the transverse direction of the rear surface of the housing 30 may be appropriately determined. For example, the length of the abutment 40 in the transverse direction of the rear surface of the housing 30 may be shorter or longer than the length of the support 50 in the transverse direction of the rear surface of the housing 30. The length of the abutment 40 in the transverse direction of the rear surface of the housing 30 and the length of the support 50 in the transverse direction of the rear surface of the housing 30 may be equal to each other.

When the mobile terminal apparatus 1 measures the biological information, the abutment 40 comes into contact with the measured part. That is, in measurement of the biological information, the abutment 40 comes into contact with, for example, the user's torso or in the vicinity thereof. As illustrated in FIG. 2B, the gyro sensor 12 is attached to the rear side of the abutment 40. In the example illustrated in FIG. 2B, the gyro sensor 12 is arranged inside the housing 30 and thus indicated by the broken lines. The abutment 40 and the gyro sensor 12 may be configured with individual members or integrally formed as the same member.

When the mobile terminal apparatus 1 measures the biological information, the support 50 comes into contact with the user at a position different from the abutment 40. The support 50 comes into contact with, for example, the user's torso at a position different from the abutment 40. The support 50 comes into contact with the user and supports an abutment state of the abutment 40 at the measured part. The mobile terminal apparatus 1 may include a plurality of supports 50. The plurality of supports 50 are linearly arranged, for example The abutment 40 and the support 50 (and the housing 30) are configured such that the movement in the measured part in contact with the abutment 40 is appropriately transmitted to the gyro sensor 12. An abutment state of the abutment 40 and the support 50 on the measured part will be described in detail later.

The mobile terminal apparatus 1 according to the embodiment of the present disclosure is not limited to have the configuration illustrated in FIGS. 2A and 2B. As described above, the mobile terminal apparatus 1 according to the embodiment may omit elements other than the essential constitutional element or include other elements, as necessary.

To measure the biological information using the mobile terminal apparatus 1 illustrated in FIGS. 2A and 2B, the user himself/herself puts the mobile terminal apparatus 1 on the user's torso by hand or the like.

Next, the measurement processing of the biological information performed by the mobile terminal apparatus 1 will be described. The mobile terminal apparatus 1 acquires the motion factor while the abutment 40 fixed to the mobile terminal apparatus 1 abuts on the measured part, and measures the biological information on the basis of the motion factor. The mobile terminal apparatus 1 may acquire the motion factor while the support 50 fixed to the mobile terminal apparatus 1 abuts on the user at a position different from the measured part.

For the measurement of the biological information, the mobile terminal apparatus 1 transitions to a state in which the measurement processing of the biological information can be performed in response to, for example, an input operation by the user. The state in which the measurement processing of the biological information can be performed refers to a state in which, for example, an application for measuring the biological information is activated. The user sets the mobile terminal apparatus 1 to the state in which the measurement processing of the biological information can be performed and causes the mobile terminal apparatus 1 to start acquiring the motion factor.

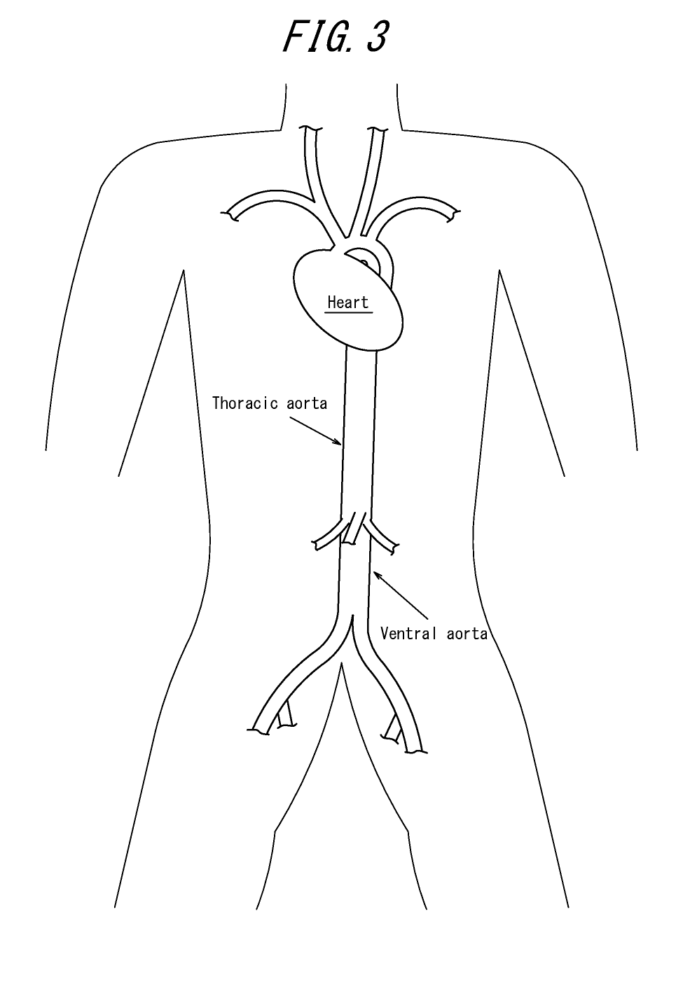

The principle on which the mobile terminal apparatus 1 measures the biological information of the user will now be further explained. The mobile terminal apparatus 1 measures the biological information on the basis of movement in the user's torso. FIG. 3 is a diagram schematically illustrating the internal structure of a human body. FIG. 3 schematically illustrates the internal structure of a part of the human body. FIG. 3 also schematically illustrates, in particular, the heart and a part of the aorta in the human body.

Blood in the human body is pumped from the heart and supplied to various parts of the human body via blood vessels. As illustrated in FIG. 3, in the human body some of the blood pumped from the heart passes through the thoracic aorta and then the ventral aorta. When the blood from the heart is delivered to the thoracic aorta or the ventral aorta, fluctuations such as expansion and contraction occur in these blood vessels. Such fluctuations travel within the body of the user and cause movement of the user's torso. Thus, when the mobile terminal apparatus 1 is pressed against the torso including the chest or abdomen of the user, the gyro sensor 12 may detect the movement of the user's torso. In this way, the gyro sensor 12 detects the motion factor caused by the movement of the user's torso.

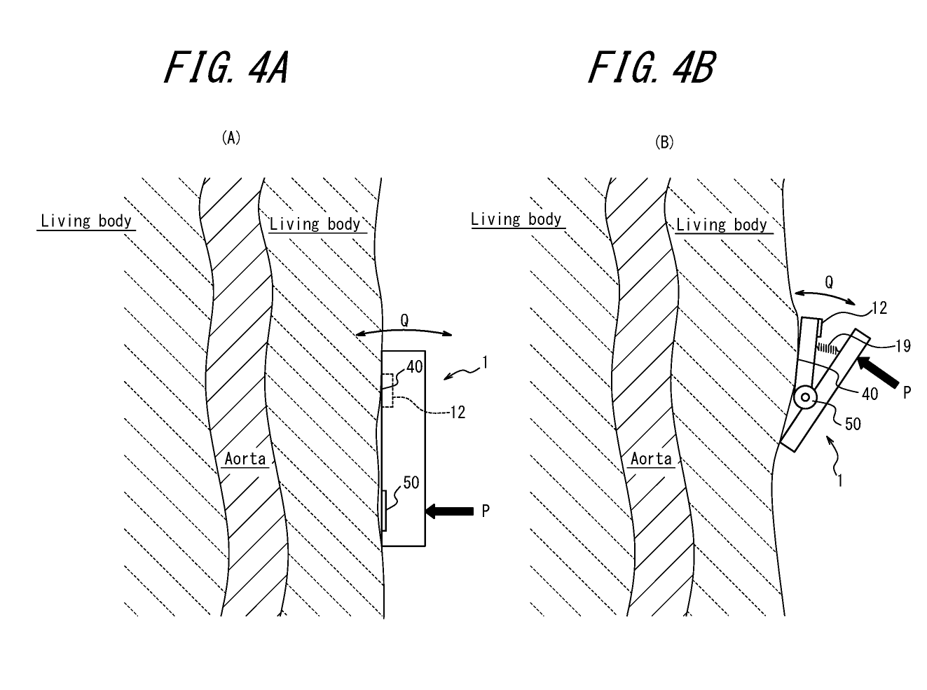

FIGS. 4A and 4B are diagrams illustrating examples of a motion factor acquiring mode of the mobile terminal apparatus 1. FIG. 4A is a diagram illustrating an example in which the mobile terminal apparatus 1 includes the gyro sensor 12 (e.g., built-in to a main body). FIG. 4B is a diagram illustrating an example in which the mobile terminal apparatus 1 does not include the gyro sensor 12 in the main body, and a member such as an external casing or attachment includes the gyro sensor 12.

FIG. 4A and FIG. 4B illustrate a cross-section of a part of a living body such as the human body that includes the aorta. FIG. 4A and FIG. 4B illustrate states in which the rear surface of the housing 30 of the mobile terminal apparatus 1 illustrated in FIGS. 2A and 2B abuts on the measured part of the living body. As illustrated in FIGS. 4A and 4B, therefore, the abutment 40 and the support 50 each abut the measured part of a surface of the living body (the skin). According to the present embodiment, the measured part on the surface of the living body is the user's torso. The aorta illustrated in FIGS. 4A and 4B may be the thoracic aorta or the ventral aorta illustrated in FIG. 3.

As illustrated in FIGS. 4A and 4B, the user presses the mobile terminal apparatus 1 against the torso to cause the mobile terminal apparatus 1 to acquire the motion factor. FIGS. 4A and 4B illustrate a contact state of the mobile terminal apparatus 1 and the user's torso, in which the abutment 40 abuts on the measured part. FIGS. 4A and 4B illustrate an acquisition state of the motion factor by the mobile terminal apparatus 1, in which the support 50 abuts the user's torso at a position different from the abutment 40.

As illustrated in FIGS. 4A and 4B, when the mobile terminal apparatus 1 is pressed against the torso in a direction indicated by the arrow P and abuts the torso, the mobile terminal apparatus 1 moves in accordance with the expansion and contraction of the blood vessel based on the user's pulse. The mobile terminal apparatus 1 is moved in such a manner that its upper end side, which is not pressed in the direction of the arrow P in the side view, rotates in the direction indicated by the arrow Q in FIG. 4A. Also, as indicated by the arrow Q in FIG. 4B, the mobile terminal apparatus 1 is moved in such a manner that its upper end side pressed in the direction of the arrow P rotates in the side view. Such a movement is usually similar to vibration caused by repetitive reciprocation in a partial rotary motion. The gyro sensor 12 of the mobile terminal apparatus 1 acquires the pulse wave of the user by detecting the movement of the mobile terminal apparatus 1. The pulse wave is a waveform acquired from the body surface and indicating temporal variations in the volume of the blood vessel caused by blood inflow.

In the mobile terminal apparatus 1 according to the present embodiment, as described above, the gyro sensor 12 detects the motion factor caused by the movement of the user's torso. In a state where the mobile terminal apparatus 1 is pressed against the user's torso, the gyro sensor 12 detects the motion factor caused by the movement of the user's torso. The controller 10 performs the measurement processing of the biological information of the user on the basis of the motion factor detected by the gyro sensor 12 as described above.

Here, the user's torso may include user's abdomen or chest. The movement of the user's torso is described as the movement caused by the fluctuation in the blood vessel of the user in FIGS. 4A and 4B by way of example, but is not limited thereto. The movement of the user's torso is not limited to the movement caused by fluctuations of the blood vessel of the user and may include at least a movement caused by the user's respiration or a movement caused by motion of the user's body. Also, the blood vessel of the user may include the aorta of the user. The aorta of the user may include at least the ventral aorta or thoracic aorta of the user. In a large blood vessel such as the aorta, a large amount of blood flows continuously. Thus, the mobile terminal apparatus 1 may measure the biological information with high accuracy and stability by using the aorta of the user as a measurement target.

As illustrated in FIG. 4B, when the gyro sensor 12 is pressed against the user's torso via an elastic member 19, the gyro sensor 12 may readily follow the movement of the user's torso. Accordingly, the mobile terminal apparatus 1 may highly accurately and stably measure the biological information. Here, the elastic member 19 may be any member that generates an elastic force and may be, for example, a spring, a rubber member, a flexible resin member, or a member that utilizes oil pressure, air pressure, or water pressure. The support 50 illustrated in FIG. 4B joins a portion of the housing that includes the gyro sensor 12 and a portion of the housing that does not include the gyro sensor 12. As illustrated in FIG. 4B, the portion of the housing that includes the gyro sensor 12 is movable with respect to the portion of the housing that does not include the gyro sensor 12 by using the support 50 as an axis.

As described above, the mobile terminal apparatus 1 illustrated in FIG. 4B may be configured such that the main body does not include the gyro sensor. In this case, an external member such as an attachment that includes the gyro sensor 12 and the abutment 40 as illustrated in FIG. 4B may be attached to the mobile terminal apparatus 1 via the support 50. In this configuration, a detection signal detected by the gyro sensor 12 may be provided to the controller 10 of the mobile terminal apparatus 1 via, for example, the support 50.

The mobile terminal apparatus 1 equipped with the gyro sensor 12 allows the user wearing clothes to measure the biological information via the clothes. That is, the mobile terminal apparatus 1 eliminates the necessity for the user to undress in order to measure the biological information. Further, the mobile terminal apparatus 1 eliminates the necessity for the user to bring the measurement apparatus into direct contact with the skin. Thus, the mobile terminal apparatus 1 enables easy measurement of the biological information.

Typically, conventional acceleration sensors are associated with high noise levels and thus are unsuitable for use as pulse wave sensors. In particular, use of a small acceleration sensor incorporated into an apparatus such as a small terminal for measurement of low frequencies around 1 Hz, such as a pulse wave and the respiration, is not common. For such purposes, a large acceleration sensor is normally required.

On the other hand, the mobile terminal apparatus 1 uses the gyro sensor 12 for the measurement of the biological information. Generally, gyro sensors performed measurement with low noise levels. The gyro sensor constantly vibrates (in the case of a vibrating gyro sensor) and may reduce the noise by virtue of its structure. Further, the mobile terminal apparatus 1 according to the present embodiment may employ a gyro sensor 12 that can be built-in to a small-scale housing 30.

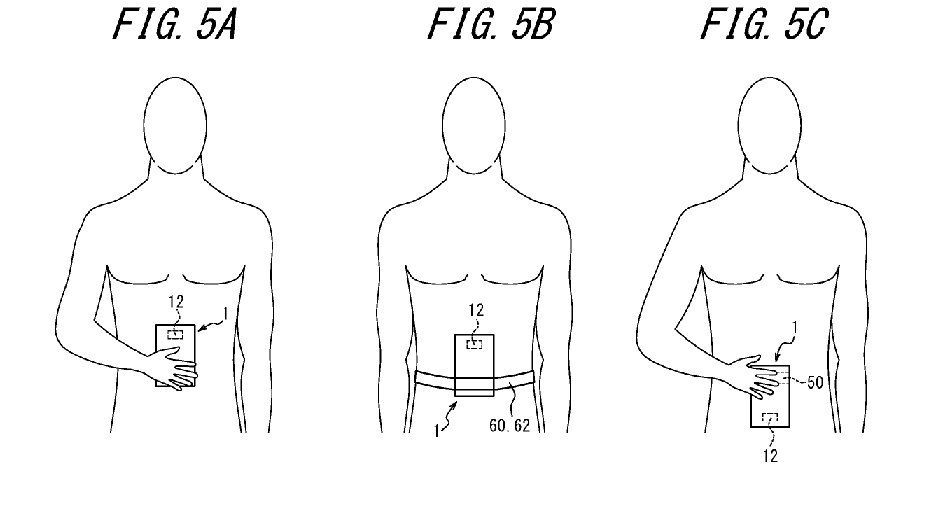

Next, a usage state of the mobile terminal apparatus 1 according to an embodiment will be described. FIGS. 5A to 5C are diagrams illustrating examples in which the biological information is measured by the mobile terminal apparatus 1. In FIGS. 5A to 5C, the gyro sensor 12 built in the mobile terminal apparatus 1 is indicated by broken lines.

FIG. 5A illustrates an example in which the biological information is measured by the mobile terminal apparatus 1 illustrated in FIGS. 2A and 2B. As illustrated in FIG. 5A, to measure the biological information with the mobile terminal apparatus 1, the user may press the abutment 40 of the mobile terminal apparatus 1 against the measured part by hand.

When the user presses the mobile terminal apparatus by hand, the user may avoid pressing the gyro sensor 12 as illustrated in FIG. 5A for better detection of fluctuations of the blood vessel by the gyro sensor 12. In this case, the user may press an area where the gyro sensor 12 is not provided, i.e., an area in the vicinity of the lower end position of the mobile terminal apparatus 1 illustrated in FIG. 2A. The support 50 illustrated in FIG. 2B is provided on a rear side of the area in the vicinity of the lower end portion of the mobile terminal apparatus 1 illustrated in FIG. 2A.

When the user presses the mobile terminal apparatus by hand, the user may change the measured part, as desired, on which the abutment 40 of the mobile terminal apparatus 1 abuts. For example, the user may move the mobile terminal apparatus 1 slightly upward to facilitate the detection of a fluctuation in the thoracic aorta. Or, the user may move the mobile terminal apparatus 1 slightly downward to facilitate the detection of a fluctuation in the ventral aorta. As described above, the user of the mobile terminal apparatus 1 may seek for a position of the measured part for better measurement of the biological information and thus highly accurately measure the biological information.

FIG. 5B illustrate an example in which a casing, a holder, or an attachment as described above which enables attachment of the mobile terminal apparatus 1 to a belt or a waistband is used. As illustrated in FIG. 5B, when the user is wearing a belt 60 or a waistband 62, the mobile terminal apparatus 1 may be attached to the belt 60 or the waistband 62 of the user via the casing, the holder, or the attachment. Such a casing, holder, or attachment may be appropriately designed to serve as an external member that enables attachment of the mobile terminal apparatus 1 to the belt 60 or the waistband 62 of the user.

This configuration eliminates the necessity for the user to press the abutment 40 of the mobile terminal apparatus 1 against the measured part for the measurement of the biological information. In this case, also, the user may change, to some extent, the measured part on which the abutment 40 of the mobile terminal apparatus 1 abuts, by adjusting a position where the belt 60 or the waistband 62 presses the mobile terminal apparatus 1. Thus, the user of the mobile terminal apparatus 1 may seek for a position of the measured part for better measurement of the biological information and highly accurately measure the biological information.

According to the present embodiment, as described above, a portion of the mobile terminal apparatus 1 may be pressed against the user's torso and, simultaneously, at least a portion other than the above portion of the mobile terminal apparatus 1 may be pressed against the belt 60 of the cloth of the user or the waistband 62. In this state, the gyro sensor 12 may detect the motion factor. The controller 10 may perform the measurement process on the basis of the motion factor detected in this manner.

FIG. 5C illustrates an example in which the mobile terminal apparatus 1 illustrated in FIG. 5A is used being upside down. In the example illustrated in FIG. 5C, as compared to the examples illustrated in FIG. 5A and FIG. 5B, the motion of the ventral aorta may be readily detected. In this case, for the measurement of the biological information, the user presses the abutment 40 of the mobile terminal apparatus 1 to the measured part by hand, or by using the belt 60 or the waistband 62.

According to the present embodiment, as described above, a portion of the mobile terminal apparatus 1 may be pressed to the lower abdomen of the user while at least a portion of the mobile terminal apparatus 1 other than the above portion may be pressed to the torso at a position closer to the head than the lower abdomen. In this state, the gyro sensor 12 may detect the motion factor. The controller 10 may perform the measurement process on the basis of the motion factor detected in this manner.

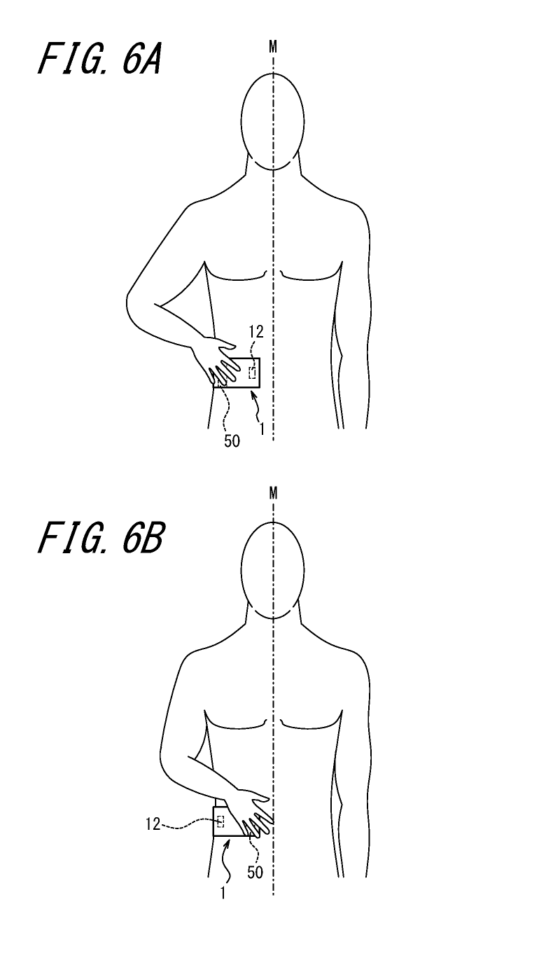

Similarly to FIGS. 5A to 5C, FIGS. 6A and 6B are diagrams illustrating other examples of the measurement of the biological information by the mobile terminal apparatus 1. In FIGS. 6A and 6B, also, the gyro sensor 12 built-in to the mobile terminal apparatus 1 is indicated by the broken lines.

As illustrated in FIG. 6A, the user may measure the biological information by turning the mobile terminal apparatus 1 sideways. In the state illustrated in FIG. 6A, when the user presses the mobile terminal apparatus 1 by hand, the user may avoid pressing the position of the gyro sensor 12 for better detection of the fluctuation in the blood vessel by the gyro sensor 12. In this case, the user may press the portion where the gyro sensor 12 is not provided, i.e., the area in the vicinity of the end portion of the mobile terminal apparatus 1 having the support 50 by hand. In this case, the gyro sensor 12 is positioned close to the center line M of the torso and may successfully detect the fluctuation in the thoracic aorta or the ventral aorta.

Or, as illustrated in FIG. 6B the mobile terminal apparatus 1 may be oriented opposite to its orientation illustrated in FIG. 6A. In this case, the gyro sensor 12 abuts on the side surface of the torso, i.e., in the vicinity of the flank. In this case, also, the user may press the portion where the gyro sensor 12 is not provided, i.e., the area in the vicinity of the end portion of the mobile terminal apparatus 1 having the support 50 by hand.

According to the present embodiment, as described above, a portion of the mobile terminal apparatus 1 may be pressed on the side surface of the user's torso while at least a portion other than the above portion of the mobile terminal apparatus 1 is pressed to a position closer to the center line M than the side surface of the user's torso. In this state, the gyro sensor 12 may detect the motion factor. The controller 10 may perform the measurement process on the basis of the motion factor detected in this manner.

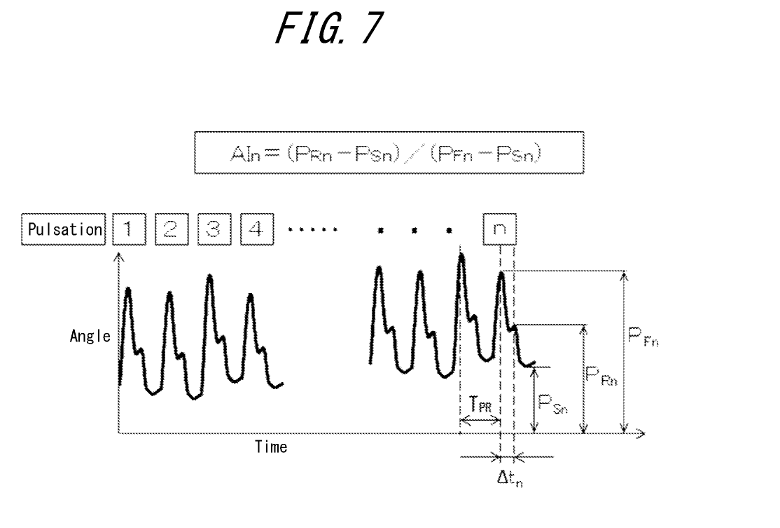

FIG. 7 is a graph illustrating an example of the pulse wave acquired from the measured part (torso) by the mobile terminal apparatus 1. In FIG. 7, the gyro sensor 12 is used as a means for detecting pulsation. FIG. 7 is a graph of the integral of the angular velocity acquired by the gyro sensor 12 serving as the angular velocity sensor. In FIG. 7, the horizontal axis represents time, and the vertical axis represents angle. The acquired pulse wave may include, for example, noise caused by movement of the user's body. Thus, a filter for removing a DC (Direct Current) component may be used to extract a pulsation component alone.

The mobile terminal apparatus 1 calculates a pulse wave index from the acquired pulse wave and measures the blood component using the index based on the pulse wave. The method to calculate the pulse wave index from the acquired pulse wave will be described with reference to FIG. 7. Pulse wave propagation is a phenomenon in which pulsation caused by blood being pumped from the heart is transmitted through the arterial wall and the blood. The pulsation transmitted by the blood pumped from the heart reaches peripheral sites of the hands and the feet as a forward wave, a portion of which is reflected at locations such as where a blood vessel branches, or where the diameter of a blood vessel changes, and returns as a reflected wave. The pulse wave index may be, for example, a Pulse Wave Velocity (PWV) of the forward wave, a magnitude P_{R} of the reflected wave of the pulse wave, a time difference Δt between the forward wave and the reflected wave of the pulse wave, or an Augmentation Index (AI) representing a ratio of the magnitude of the forward wave to the magnitude of the reflected wave.

The pulse wave illustrated in FIG. 7 corresponds to n-beats of the user's pulse, where n is an integer of at least 1. The pulse wave is a combined wave in which the forward wave caused by the blood pumped from the heart overlaps with the reflected wave caused by branches of the blood vessel or portions where the diameter of the blood vessel changes. In FIG. 7, P_{Fn} represents a magnitude of a peak of the pulse wave caused by the forward wave of each pulse, and P_{Rn} represents the magnitude of a peak of the pulse wave caused by the reflected wave of each pulse. P_{Sn} represents a minimum value of the pulse wave of each pulse. In FIG. 7, also, T_{PR} represents an interval between the peaks of the pulses.

The pulse wave index quantifies information obtained from the pulse wave. An example of a pulse wave index is PWV, which is calculated using a difference in propagation time of pulse waves measured at two measured parts such as the upper arm and the ankle, and a distance therebetween. In particular, the PWV is calculated by synchronously acquiring pulse waves at two points of an artery (e.g., at the upper arm and the ankle) and dividing the distance (L) between the two points by a time difference (PTT) of the pulse waves at the two points. A further example of the pulse wave index is the magnitude P_{R} of the reflected wave, which may be calculated as the magnitude P_{Rn} of the peak of the pulse wave caused by the reflected wave or a mean value P_{Rave} of the n values of P_{Rn}. A further example of a pulse wave index is the time difference Δt between the forward wave and the reflected wave of the pulse wave, which may be calculated as the time difference Δtn of a given pulse or a mean value Δtₐᵥₑ of the n values of Δtn. A further example of a pulse wave index is the AI, which is obtained by dividing the magnitude of the reflected wave by the magnitude of the forward wave and expressed as AIₙ=(P_{Rn}−P_{Sn})/(P_{Fn}−P_{Sn}). AIₙ represents the AI of each pulse wave. As a pulse wave index, AI may, for example be obtained by measuring the pulse wave for several seconds and calculating a mean value AIₐᵥₑ of the AIₙ (n is an integer of at least 1) of each pulse.

The PWV, the magnitude PR of the reflected wave, the time difference Δt between the forward wave and reflected wave, and the AI vary depending on hardness of the blood vessel wall and thus may be used for estimating a state of arteriosclerosis. For example, when the vessel wall is hard, the pulse wave velocity PWV increases. For example, when the vessel wall is hard, the magnitude P_{R} of the reflected wave increases. For example, when the vessel wall is hard, the time difference Δt between the forward wave and the reflected wave decreases. For example, when the vessel wall is hard, the AI increases. Further, by using these pulse wave indices as described above, the mobile terminal apparatus 1 may estimate the state of arteriosclerosis and the blood fluidity (viscosity). In particular, the mobile terminal apparatus 1 may estimate a change in the blood fluidity from a change in the pulse wave index acquired at the same measured part of the same user during a time period over which there is substantially no change in the state of arteriosclerosis (e.g. within a few days). Here, the blood fluidity indicates a degree of ease of the blood flow. For example, when the blood fluidity is low, the pulse wave velocity PWV decreases. For example, when the blood fluidity is low, the magnitude P_{R} of the pulse wave decreases. For example, when the blood fluidity is low, the time difference Δt between the forward wave and the reflected wave increases. For example, when the blood fluidity is low, the AI decreases.

According to an embodiment, the mobile terminal apparatus 1 calculates, by way of example, the pulse wave velocity (PWV), the magnitude P_{R} of the reflected wave, the time difference Δt between the forward wave and the reflected wave, or the AI as the pulse wave index. However, the pulse wave index is not limited thereto. For example, the mobile terminal apparatus 1 may use a posterior systolic blood pressure as the pulse wave index.

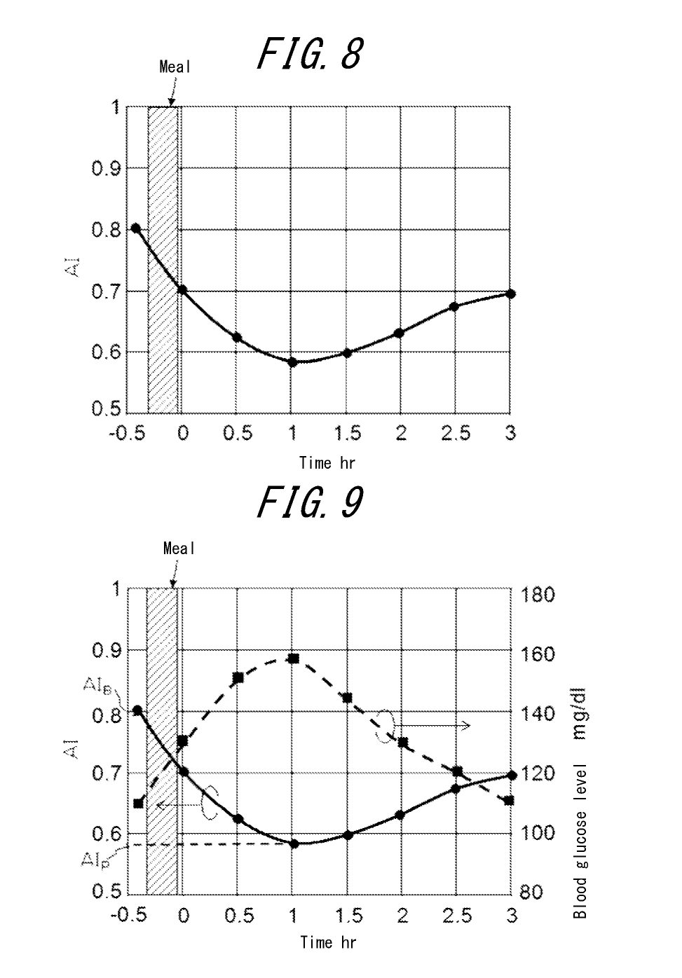

FIG. 8 is a graph illustrating temporal variation in the calculated AI. According to an embodiment, the pulse wave was acquired for approximately 5 seconds by the mobile terminal apparatus 1 equipped with the angular velocity sensor. The controller 10 calculated the AI of each pulse from the obtained pulse wave and further calculated a mean value AIₐᵥₑ of the AI. According to the present embodiment, the mobile terminal apparatus 1 acquired the pulse wave at a plurality of timings before and after a meal and calculated the mean value of the AI (simply “AI” below) as one example of the pulse wave index. In FIG. 8, the horizontal axis indicates elapsed time, with a first measurement time after the meal set as 0. In FIG. 8, the vertical axis indicates the AI calculated from the pulse wave acquired at the corresponding time.

The mobile terminal apparatus 1 acquired the pulse wave before the meal, immediately after the meal, and every 30 minutes after the meal, and calculated a plurality of AI based on the pulse wave at each time. The AI calculated from the pulse wave obtained before the meal was approximately 0.8. As compared with the AI before the meal, the AI immediately after the meal was lower, and the AI reached its lowest value approximately 1 hour after the meal. Then, the AI gradually increased in the three hours after the meal, until the completion of the measurement.

The mobile terminal apparatus 1 can estimate the change in the blood fluidity from the change in the calculated AI. For example, the blood fluidity becomes low when the red blood cells, the white blood cells, and the platelets are solidified, or when the adhesiveness increases. For example, the blood fluidity becomes low when the plasma content in the blood decreases. The blood fluidity changes depending on the state of health of the user such as states of the glucose metabolism and the lipid metabolism described below, heat stroke, dehydration, hypothermia, and the like. The mobile terminal apparatus 1 according to the present embodiment enables the user to know a change in his/her blood fluidity before the state of health of the user becomes severe. From the changes in the AI before and after the meal as illustrated in FIG. 8, it may be estimated that the blood fluidity decreased after the meal, became minimum in approximately 1 hour after the meal, and then gradually increased thereafter. The mobile terminal apparatus 1 may inform the user of a state in which the blood fluidity is low and a state in which the blood fluidity is high. For example, the mobile terminal apparatus 1 may determine the state in which the blood fluidity is low and the state in which the blood fluidity is high, on the basis of a mean of the AI of the actual age of the user. The mobile terminal apparatus 1 may determine that the blood fluidity is high when the calculated AI is larger than the mean value, or that the blood fluidity is low when the calculated AI is smaller than the mean value. For example, the mobile terminal apparatus 1 may determine the state in which the blood fluidity is low and the state in which the blood fluidity is high, on the basis of the AI before a meal. The mobile terminal apparatus 1 may estimate the degree of the state in which the blood fluidity is low by comparing the AI after a meal with the AI before the meal. For example, the mobile terminal apparatus 1 may use the AI before a meal, that is, the AI of the user having an empty stomach as an index of a vascular age (blood vessel hardness) of the user. For example, by calculating a change amount of the calculated AI on the basis of the AI before the meal, i.e., the AI when the user's stomach is empty, the mobile terminal apparatus 1 may reduce an estimation error due to the vascular age (blood vessel hardness) of the user. The mobile terminal apparatus 1 may more accurately estimate changes in the blood fluidity.

FIG. 9 is a graph illustrating the calculated AI and results of measurement of a blood glucose level. The method for acquiring the pulse wave and the method for calculating the AI are similar to those of the embodiment illustrated in FIG. 8. In FIG. 9, the vertical axis on the right side represents the blood glucose level, and the vertical axis on the left side represents the calculated AI. In FIG. 9, a solid line represents the AI calculated from the acquired pulse wave, and a broken line represents a measured blood glucose level. The blood glucose level was measured immediately after the pulse wave was acquired. The blood glucose level was measured using the “Medi-Safe Fit” blood glucose measurement device produced by TERUMO CORPORATION. As compared with the blood glucose level before the meal, the blood glucose level immediately after the meal increased by approximately 20 mg/dl. The blood glucose level reached a maximum at approximately 1 hour after the meal. Then, the blood glucose level gradually decreased until the measurement was completed and, at approximately 3 hours after the meal, became substantially equal to the blood glucose level before the meal.

As illustrated in FIG. 9, the blood glucose levels before and after the meal are negatively correlated with the AI calculated from the pulse wave. When the blood glucose level is high, sugar in the blood causes massing of the red blood cells and the platelets, or increases the viscosity of the blood. As a result, the blood fluidity may decrease. The decrease in the blood fluidity may reduce the pulse wave velocity PWV. The decrease in the pulse wave velocity PWV may cause an increase in the time difference Δt between the forward wave and the reflected wave. The increase in the time difference Δt between the forward wave and the reflected wave may cause a decrease in the magnitude P_{R} of the reflected wave with respect to the magnitude P_{F} of the forward wave. The decrease in the magnitude P_{R} of the reflected wave with respect to the magnitude P_{F} of the forward wave may cause a decrease in the AI. The AI within several hours after the meal (3 hours according to the present embodiment) is correlated with the blood glucose level. Therefore, a change in the blood glucose level of the user may be inferred from the change in the AI. Also, by measuring the blood glucose level of the user in advance and obtaining a correlation thereof with the AI, the mobile terminal apparatus 1 may estimate the blood glucose level of the user based on the calculated AI.

The mobile terminal apparatus 1 may estimate a state of the user's glucose metabolism on the basis of the time at which a first detected minimum extreme value AI_{P} of the AI occurs after the meal. The mobile terminal apparatus 1 estimates, for example, the blood glucose level indicative of the state of the glucose metabolism. As an example of estimating the state of the glucose metabolism, the mobile terminal apparatus 1 may infer that the user has glucose metabolism disorder (the user is a diabetic) when, for example, a first detected minimum extreme value AI_{P} of the AI after the meal is detected after a predetermined time period (e.g., approximately 1.5 hours from the meal) or longer.

The mobile terminal apparatus 1 may estimate the state of the user's glucose metabolism on the basis of a difference (AI_{B}−AI_{P}) between AI_{B} representing the AI before the meal and the first detected minimum extreme value AI_{P} of the AI after the meal. As an example of estimating the state of the glucose metabolism, the mobile terminal apparatus 1 may infer that the user has glucose metabolism disorder (the user is a postprandial hyperglycemia patient) when (AI_{B}−AI_{P}) is greater than or equal to a predetermined value (e.g., 0.5 or higher).

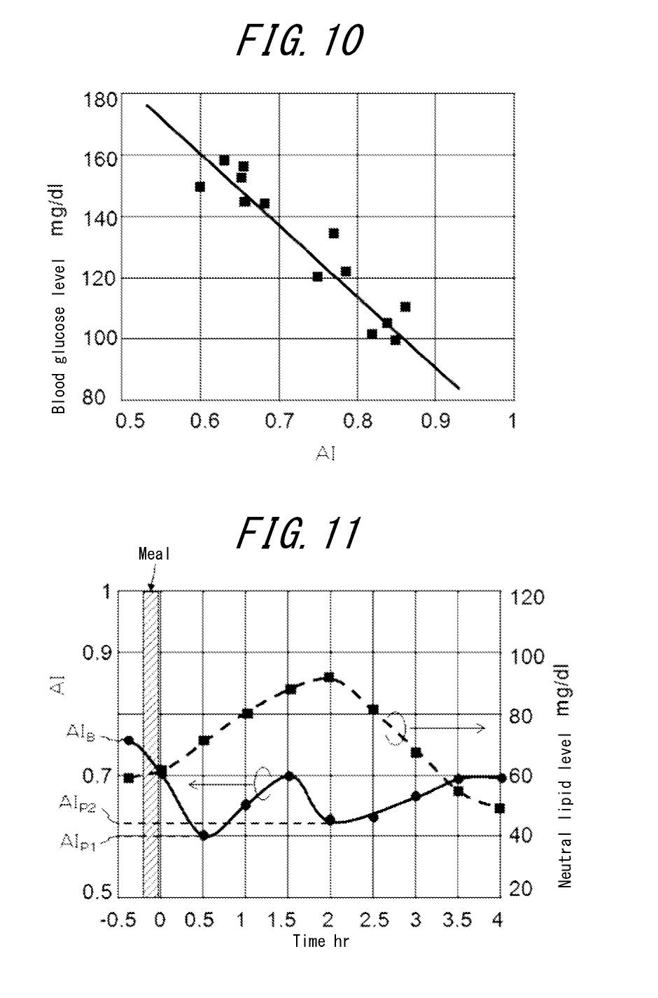

FIG. 10 is a graph illustrating a relationship between the calculated AI and the blood glucose level. The calculated AI and the blood glucose level were acquired within 1 hour after a meal, when the blood glucose level varies greatly. The data of FIG. 10 includes a plurality of different data points for the same user after a meal. As illustrated in FIG. 10, the calculated AI and the blood glucose level are negatively correlated. The correlation coefficient of the calculated AI and the blood glucose level is at least 0.9, indicating high correlation. For example, by acquiring the correlation of the calculated AI and the blood glucose level as illustrated in FIG. 10 from each user in advance, the mobile terminal apparatus 1 may estimate the user's blood glucose level from the calculated AI.

FIG. 11 is a graph illustrating the calculated AI and results of the measurement of a neutral lipid level. The method for acquiring the pulse wave and the method for calculating the AI are the same as those of the embodiment illustrated in FIG. 8. In FIG. 11, the vertical axis on the right side represents the neutral lipid level in the blood, and the vertical axis on the left side indicates the AI. In FIG. 11, the solid line represents the AI calculated from the obtained pulse wave, and the broken line represents a measured triglyceride level. The neutral lipid level was measured immediately after the pulse wave was acquired. The neutral lipid level was measured using the “Pocket Lipid” lipid measuring device produced by Techno Medica Co., Ltd. As compared with the neutral lipid level before the meal, the maximum extreme value of the neutral lipid level after the meal rose by approximately 30 mg/dl. The neutral lipid level reached the maximum extreme value at approximately 2 hours after the meal. Subsequently, the neutral lipid level gradually decreased until the measurement was completed and, at approximately 3.5 hours after the meal, became substantially equal to the neutral lipid level before the meal.

In contrast, the minimum extreme values of the calculated AI were a first minimum extreme value AI_{P1} detected at approximately 30 minutes after the meal, and a second minimum extreme value AI_{P2} detected at approximately 2 hours after the meal. It can be inferred that the first minimum extreme value AI_{P1} detected at approximately 30 minutes after the meal was caused by the influence of the blood glucose level after the meal as described above. The second minimum extreme value AI_{P2} detected at approximately 2 hours after the meal is substantially coincident with the maximum extreme value of the neutral lipid level detected at approximately 2 hours after the meal. From this, it can be inferred that the second minimum extreme value AI_{P2} detected after a predetermined time period from the meal is due to the effect of the neutral lipids. It can be understood that the neutral lipid levels before and after the meal, similarly to the blood glucose level, have a negative correlation with the AI calculated from the pulse wave. In particular, the minimum extreme value AI_{P2} of the AI detected after the predetermined time period (after 1.5 hours according to the present embodiment) from the meal is correlated with the neutral lipid level. Therefore, the change in the user's neutral lipid level can be estimated from the change in the AI. Also, by measuring the user's neutral lipid level in advance and determining a correlation with the AI, the mobile terminal apparatus 1 may estimate the neutral lipid level of the user from the calculated AI.

The mobile terminal apparatus 1 can estimate the state of the user's lipid metabolism on the basis of the time at which the second minimum extreme value AI_{P2} is detected after the predetermined time from the meal. The mobile terminal apparatus 1 estimates, for example, a lipid level as the state of the lipid metabolism. As an example, the mobile terminal apparatus 1 may infer that the user has abnormal lipid metabolism (the user is a hyperlipidemia patient) when the second minimum extreme value AI_{P2} is detected after the predetermined time or longer (e.g., more than 4 hours) from the meal.

The mobile terminal apparatus 1 can estimate the state of the user's lipid metabolism on the basis of a difference (AI_{B}−AI_{P2}) between AI_{B} representing the AI before the meal and the second minimum extreme value AI_{P2} detected after the predetermined time period from the meal. As an example, the mobile terminal apparatus 1 can infer that the user's lipid metabolism is abnormal (the user is a postprandial hyperlipidemia patient), when, for example, the difference (AI_{B}−AI_{P2}) is equal to or greater than 0.5.

Also, from the results of the measurement illustrated in FIG. 9 to FIG. 11, the mobile terminal apparatus 1 according to the present embodiment may estimate the state of the user's glucose metabolism on the basis of the first minimum extreme value AI_{P1}, detected earliest after the meal, and the occurrence time thereof. Further, the mobile terminal apparatus 1 according to the present embodiment can estimate the state of the user's lipid metabolism on the basis of the second minimum extreme value AI_{P2}, detected after the predetermined time period from the detection of the first minimum extreme value AI_{P1}, and the occurrence time thereof.

Although, according to the present embodiment, the neutral lipid level is acquired for the estimation of the lipid metabolism, the estimation of the lipid metabolism is not limited thereto. The lipid level estimated by the mobile terminal apparatus 1 includes, for example, total cholesterol level, High Density Lipoprotein (HDL) cholesterol level, Low Density Lipoprotein (LDL) cholesterol level, and so on. These lipid levels exhibit tendencies similar to the above described case of neutral lipids.

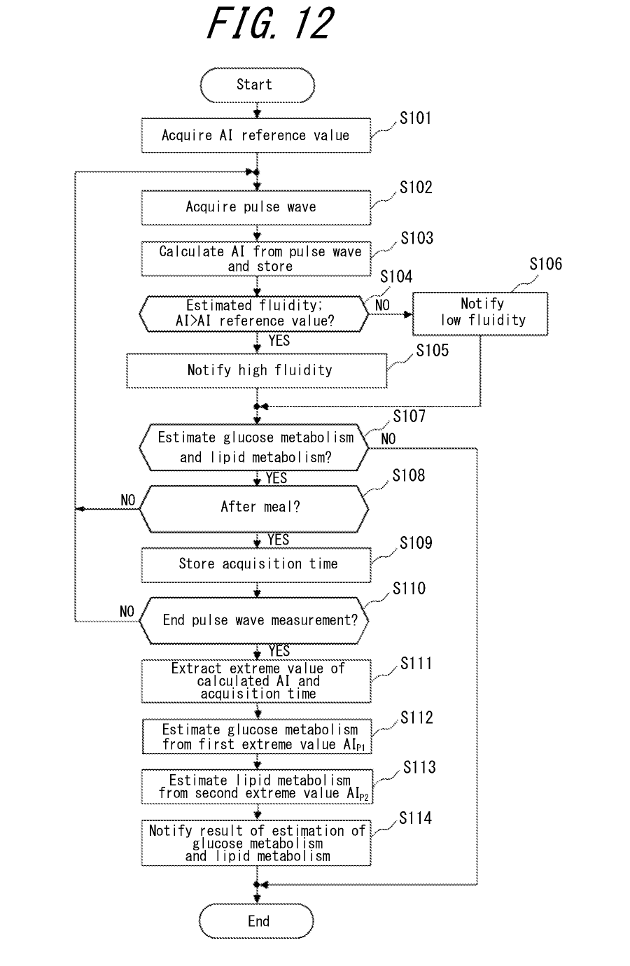

FIG. 12 is a flowchart illustrating a process for estimating the blood fluidity and the states of the glucose metabolism and the lipid metabolism on the basis of the AI. Referring to FIG. 12, the process by which the mobile terminal apparatus 1 according to an embodiment estimates the blood fluidity and the states of the glucose metabolism and the lipid metabolism based on the AI will be described.

As illustrated in FIG. 12, the mobile terminal apparatus 1 acquires an AI reference value of the user as an initial setting (step S101). The AI reference value may be an average AI estimated from the user's age or the AI acquired in advance from the user with an empty stomach. The mobile terminal apparatus 1 may also use the AI determined to be before the meal in steps S102 to S108 or the AI calculated immediately before the measurement of the pulse wave as the AI reference value. In this case, the mobile terminal apparatus 1 executes step S101 after steps S102 to S108.

Subsequently, the mobile terminal apparatus 1 acquires the pulse wave (step S102). For example, the mobile terminal apparatus 1 determines whether a pulse wave with at least a predetermined amplitude is obtained in a predetermined measurement time (e.g., 5 seconds). When a pulse wave with at least the predetermined amplitude is acquired, the mobile terminal apparatus 1 proceeds to step S103. When a pulse wave with at least the predetermined amplitude is not acquired, the mobile terminal apparatus 1 repeats step S102 (these steps are not illustrated). At step S102, upon detection of, for example, a pulse wave with at least the predetermined amplitude, the mobile terminal apparatus 1 autonomously acquires the pulse wave.

The mobile terminal apparatus 1 calculates the AI serving as the pulse wave index from the pulse wave obtained at step S102 and stores the calculated AI in the memory 20 (step S103). The mobile terminal apparatus 1 may obtain the AI by calculating the average AIₐᵥₑ from the AIₙ (n is an integer of 1 to n) of a predetermined number of pulses (e.g., 3 pulses). Alternatively, the mobile terminal apparatus 1 may calculate the AI of a specific pulse.

The AI may be calculated by performing a correction using, for example, a pulse rate, a pulse pressure (P_{F}−P_{S}), body temperature, temperature of a detection part, and so on. It is known that there is a negative correlation between the pulse and the AI and between the pulse pressure and the AI, and also that there is a positive correlation between the temperature and the AI. When performing the correction, for example, the mobile terminal apparatus 1 calculates the pulse rate and the pulse pressure in addition to the AI at step S103. For example, the mobile terminal apparatus 1 may be equipped with a temperature sensor mounted on the sensor unit 130 to acquire temperature of the measured part when the pulse wave is obtained at step S102. The mobile terminal apparatus 1 corrects the AI by substituting the obtained pulse rate, pulse pressure, and temperature into a correction equation prepared in advance.

Next, the mobile terminal apparatus 1 compares the AI reference value obtained at step S101 with the AI calculated at step S103 and estimates the blood fluidity of the user (step S104). When the calculated AI is greater than the AI reference value (in the case of YES), it is inferred that the blood fluidity is high. In this case, the mobile terminal apparatus 1 notifies that, for example, the blood fluidity is high (step S105). When the calculated AI is not greater than the AI reference value (in the case of NO), it is inferred that the blood fluidity is low. In this case, the mobile terminal apparatus 1 notifies that, for example, the blood fluidity is low (step S106).

Next, the mobile terminal apparatus 1 confirms with the user whether to estimate the states of the glucose metabolism and the lipid metabolism (step S107). When the states of the glucose metabolism and the lipid metabolism are not to be estimated at step S107 (in the case of NO), the mobile terminal apparatus 1 ends the process. When the states of the glucose metabolism and the lipid metabolism are to be estimated at step S107 (in the case of YES), the mobile terminal apparatus 1 confirms whether the calculated AI was acquired before or after the meal (step S108). When the calculated AI was not acquired after the meal (i.e., obtained before the meal) (in the case of NO), the mobile terminal apparatus 1 returns to step S102 and acquires the next pulse wave. When the calculated AI was acquired after the meal (in the case of YES), the mobile terminal apparatus 1 stores the time at which the pulse wave corresponding to the calculated AI was acquired (step S109). Then, when the pulse wave is obtained (in the case of NO at step S110), the mobile terminal apparatus 1 returns step S102 and obtains the next pulse wave. To end the measurement of the pulse wave (in the case of YES at step S110), the mobile terminal apparatus 1 proceeds to step S111 and following steps to estimate the state of the user's glucose metabolism and lipid metabolism.

Next, the mobile terminal apparatus 1 extracts the minimum extreme value and time corresponding thereto from a plurality of AI calculated at step S104 (step S111). For example, in the case of the AI indicated by the solid line in FIG. 11 being calculated, the mobile terminal apparatus 1 extracts the first minimum extreme value AI_{P1} occurring at approximately 30 minutes after the meal and the second minimum extreme value AI_{P2} occurring at approximately 2 hours after the meal.

Next, the mobile terminal apparatus 1 estimates the state of the user's glucose metabolism from the first minimum extreme AI_{P1} and the time corresponding thereto (step S112). Also, the mobile terminal apparatus 1 estimates the state of the user's lipid metabolism from the second minimum extreme AI_{P2} and the time corresponding thereto (step S113). Examples of the estimation of the states of the user's glucose metabolism and lipid metabolism are similar to those of FIG. 11 and thus are omitted herein.

Next, the mobile terminal apparatus 1 notifies of the results of the estimation at steps S112 and S113 (step S114) and ends the process illustrated in FIG. 12. The audio output interface 16 may issue a notification such as “normal glucose metabolism”, “possible abnormal glucose metabolism”, “normal lipid metabolism”, “possible abnormal lipid metabolism”. The audio output interface 16 may issue advice such as “see a doctor”, “improve your diet”, and so on. Then, the mobile terminal apparatus 1 ends the process illustrated in FIG. 12.

As described above, the mobile terminal apparatus 1 may include the audio output interface 16 configured to output a voice. In place of or in addition to the notification sound output from the audio output interface 16 as described above, a notification may be displayed on the display 14. As described above, the mobile terminal apparatus 1 may include the display 14 configured to display information associated with the measurement process performed by the controller 10. The audio output interface 16 may output a sound indicating that the gyro sensor 12 is detecting a motion factor. This enables the user to be easily and reliably notified that the gyro sensor 12 of the mobile terminal apparatus 1 is correctly detecting the motion factor.

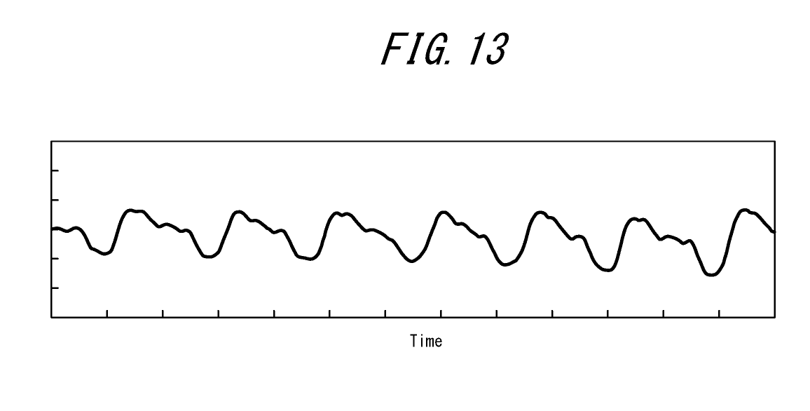

The mobile terminal apparatus 1 may also measure, as the biological information, a respiration state of the user on the basis of the motion factor. FIG. 13 is a graph illustrating an example of a respiration waveform acquired by the sensor. As illustrated in FIG. 13, the respiration waveform periodically makes peaks and valleys in accordance with the respiration of the user. The mobile terminal apparatus 1 may measure, for example, a respiration rate of the user per unit time on the basis of the respiration waveform.

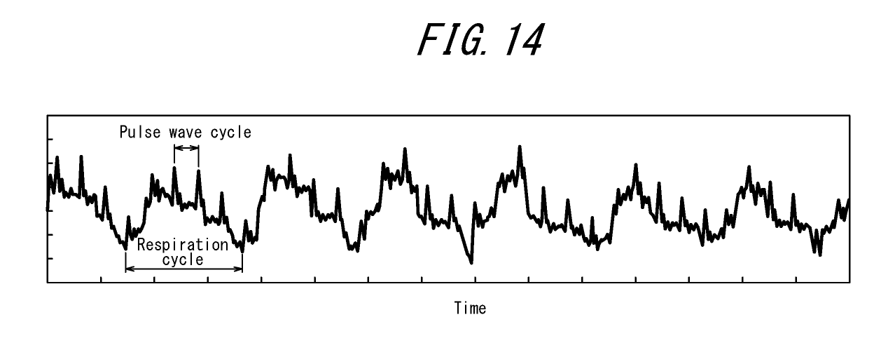

FIG. 14 is a graph illustrating an example of a synthesized waveform of a pulse wave and the respiration acquired by a sensor. When the mobile terminal apparatus 1 abuts on, for example, the abdomen serving as the measured part, the synthesized waveform of the pulse wave and the respiration as illustrated in FIG. 14 by way of example may be acquired. The mobile terminal apparatus 1 may extract a pulse wave cycle and a respiration cycle from the synthesized waveform on the basis of, for example, peak intervals or the like, and calculate the biological information such as the pulse wave and the respiration rate from the pulse wave cycle and the respiration cycle.

As described above, the biological information measured by the mobile terminal apparatus 1 may include information indicating at least one of the pulse wave, the pulse, the respiration, the heartbeat, the pulse wave velocity, and the blood flow of the user.

Further, the controller 10 may estimate the information indicating at least one of a physical condition, drowsiness, sleep, a wakening state, a psychological state, a physical state, emotion, a psychosomatic state, a mental state, an autonomic nervous state, a stress state, consciousness, blood components, a sleeping state, a respiration state, and a blood pressure of the user, on the basis of the biological information measured by the mobile terminal apparatus 1. Here, the “psychosomatic state” of the user may be, for example, presence or absence of symptoms such as heat stroke, fatigue degree, altitude sickness, diabetes, metabolic syndrome, degree of these symptoms, or presence or absence of signs of these symptoms. Also, the blood components may be the neutral fat, the blood glucose level, and the like.

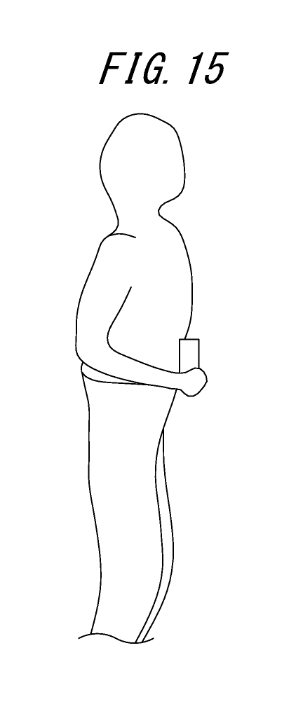

The user may use the mobile terminal apparatus 1 described above in a supine position, for example That is, in a state in which the application for measuring the biological information is activated on the mobile terminal apparatus 1, the user may perform the measurement of the biological information by pressing the mobile terminal apparatus 1 against the torso in a supine position as illustrated in FIG. 15 by way of example The user may use the mobile terminal apparatus 1 in a supine position at bedtime, for example. When the user uses the mobile terminal apparatus 1 to measure the biological information at bedtime, the mobile terminal apparatus 1 may have a function for inducing sleepiness of the user. As such, the mobile terminal apparatus 1 may have a sleep-inducing function in addition to the function of measuring the biological information. That is, the mobile terminal apparatus 1 may perform the sleep-inducing operation while performing the measurement of the biological information. The sleep-inducing operation may be executed as a part of the measurement processing of the biological information.

Here, an example of the sleep-inducing function will be described in detail. People have a respiration rhythm that tends to induce sleepiness. When people are breathing according to the respiration rhythm that tends to induce sleepiness, the parasympathetic nervous system is more active and a relaxed feeling is realized. Generally, the cycle of the respiration rhythm that tends to induce sleepiness is longer than the cycle of the respiration rhythm when people are awake. The sleep-inducing function of the mobile terminal apparatus 1 according to the present embodiment guides the respiration cycle of the user to the respiration rhythm that tends to induce sleepiness.

The mobile terminal apparatus 1 may execute the sleep-inducing function by, for example, outputting to guide the respiration rhythm of the user to the respiration rhythm that tends to induce sleepiness (hereinafter, also referred to as “a target respiration rhythm”). The target respiration rhythm is a guiding target respiration rhythm of the sleep-inducing function. The target respiration rhythm may be input, for example, as a target respiration cycle.

In inducing sleepiness, the controller 10 calculates a current respiration rhythm (e.g., a cycle) of the user on the basis of acquired biological information. The controller 10 calculates a difference between the target respiration rhythm and the calculated current respiration rhythm. The controller 10 determines an output pattern to be output to the user on the basis of the calculated difference. The controller 10 outputs in the determined output pattern. The output may be performed in any way that can be recognized by the user including, for example, a sound and a vibration.

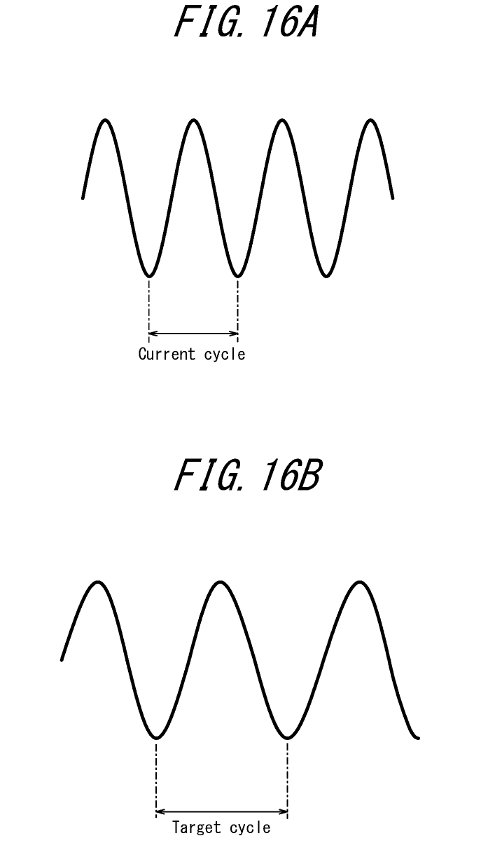

FIGS. 16A and 16b are diagrams schematically illustrating examples of respiration rhythms. FIG. 16A illustrates the current respiration cycle of the user, and FIG. 16B illustrates the target respiration cycle. As illustrated in FIGS. 16A and 16B, when the current respiration cycle is shorter than the target respiration cycle, the controller 10 calculates a difference between these cycles. The controller 10 determines an output cycle to be presented to the user, on the basis of the calculated difference. The output cycle is a virtual respiration cycle to be presented to the user. For example, at current time, the controller 10 outputs in the same cycle as the current respiration cycle of the user as illustrated in FIG. 16A and determines an output pattern (cycle) that gradually lengthens over time. For example, when a target time for the user to fall asleep is set in advance, the controller 10 determines the output pattern (cycle) to lengthen over time such that the output is performed at the target cycle as illustrated in FIG. 16B at the target time. The controller 10 performs the output in the determined output pattern. That is, the output from the mobile terminal apparatus 1 gradually lengthens over time. The output may be, for example, a sound or a vibration. The user breathes in synchronization with the output sound or vibration. By breathing following a change in the output pattern of the mobile terminal apparatus 1, the user may gradually bring the respiration cycle closer to the target cycle. Accordingly, the respiration cycle of the user approaches the cycle that tends to induce sleepiness. In this way, the mobile terminal apparatus 1 may induce sleepiness.

According to the an embodiment, the mobile terminal apparatus 1 may autonomously start and/or stop the measurement processing of the biological information when a predetermined condition is met in a state where the application for measuring the biological information is activated. Such autonomous start and/or stop of the measurement processing of the biological information eliminates the necessity for the user to perform an input operation to start and/or stop the measurement process. In this way, the mobile terminal apparatus 1 may reduce the inconvenience of the input operation to start and/or stop the measurement process.

Here, the predetermined condition for starting and/or stopping the measurement processing of the biological information will be described. In the mobile terminal apparatus 1, the controller 10 may start the measurement process when a first condition is met. The first condition is a condition for starting the measurement process. The first condition may be a condition for starting a part of the measurement process. Here, starting the measurement process may include, for example, resuming the measurement process after a pause. When the first condition is met, the controller 10 may start, for example, at least a part of the measurement process. For example, the controller 10 may start performing the sleep-inducing function when the first condition is met.

The first condition may be, for example, start of the detection of the motion factor. For example, when the user activates the application for measuring the biological information on the mobile terminal apparatus 1 and presses the mobile terminal apparatus 1 against the measured part of the user in a supine position, the motion factor is detected by the gyro sensor 12. The controller 10 may start the measurement process upon determining that the motion factor is detected.

The first condition may be elapse of a predetermined time period (e.g. several seconds) after the detection of the motion factor. For example, immediately after the start of the detection of the motion factor, the motion factor may not be stable due to, for example, a movement of the user adjusting the position to press the mobile terminal apparatus 1. By starting the measurement process after a predetermined time period subsequent to the start of the detection of the motion factor, the controller 10 may acquire a stable motion factor.

The first condition may be continuous detection of the motion factor for a predetermined time period (e.g., several seconds). For example, immediately after the start of the detection of the motion factor, the motion factor may not be stable due to, for example, the movement of the user adjusting the position to press the mobile terminal apparatus 1. When the user fixes the position to press the mobile terminal apparatus 1, the motion factor is likely to be stably detected. By starting the measurement process after the motion factor is continuously detected for a predetermined time, the controller 10 may acquire a stable motion factor.

The first condition may be transition of a cycle of the motion factor to a stable state from a disturbed state. For example, immediately after the start of the detection of the motion factor, the motion factor may not be stable due to, for example, the movement of the user adjusting the position to press the mobile terminal apparatus 1. In this case, for example, the cycle of the motion factor is unstable. When the user fixes the position to press the mobile terminal apparatus 1, the cycle of the motion factor is likely to become stable. By starting the measurement process upon determination that the cycle of the motion factor has transitioned to a stable state, the controller 10 may acquire a stable motion factor.

The first condition may be transition of the cycle of the motion factor to a disturbed state from a stable state. For example, a stable state of the cycle of the motion factor is likely to mean that the user is breathing at predetermined intervals. On the other hand, when the respiration rhythm of the user is disturbed, the cycle of the motion factor is unstable and disturbed. In some cases, measuring the biological information of the user in this state may be useful to understand a condition of the user. As such, the controller 10 may start the measurement process when determining that the cycle of the motion factor has transitioned to a disturbed state from a stable state.

The first condition is not limited to the above examples but may include other conditions. Also, the first condition may include any appropriate combination of the above example conditions and/or other conditions.

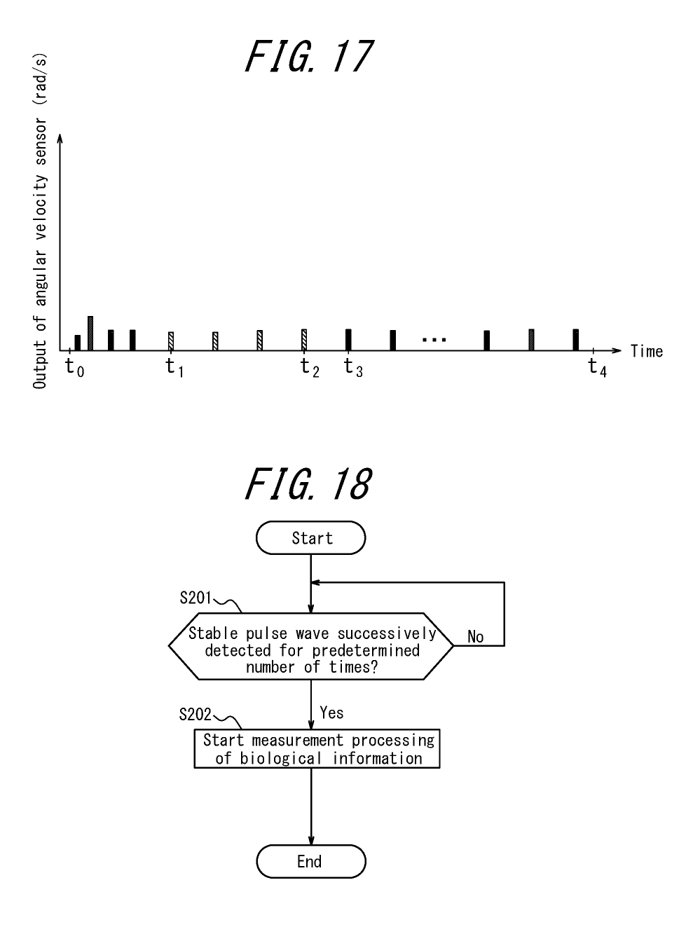

Here, a process to start the measurement process performed by the mobile terminal apparatus 1 will be described with reference to FIG. 17 and FIG. 18. FIG. 17 is a diagram schematically illustrating the motion factor acquired by the mobile terminal apparatus 1. FIG. 18 is a flowchart illustrating an example of the process to start the measurement processing of the biological information. Here, an example in which the first condition is the shift of the cycle of the motion factor to a stable state from a disturbed state will be described in detail.

In FIG. 17, the horizontal axis represents time, and the vertical axis schematically represents the motion factor, i.e., output (rad/sec) based on the pulse wave by the angular velocity sensor serving as the gyro sensor 12. In FIG. 17, the output of the angular velocity sensor indicates only a peak of each pulse wave.

For example, it is assumed that the user starts pressing the mobile terminal apparatus 1 against the measured part in a supine position at time t₀. In the mobile terminal apparatus 1, the controller 10 detects the output of the gyro sensor 12. For a predetermined time period (from time t₀ to time t₁ in FIG. 17) subsequent to the start of the measurement, the output of the gyro sensor 12 is unstable due to, for example, the movement of the user adjusting the position to bring the abutment 40 into contact with the measured part. The biological information may not be accurately acquired during this period.

The controller 10 determines whether the first condition is met, that is, whether the cycle of the motion factor has transitioned to the stable state from the unstable state. For example, by determining that stable pulse waves are detected consecutively for a predetermined number of times, the controller 10 determines that the cycle of the motion factor has transitioned to a stable condition from an unstable condition (step S201 in FIG. 18). The predetermined number of times is four in the example illustrated in FIG. 17 but is not limited thereto. Also, the stable pulse wave refers to a pulse wave having variations in the peak output and/or variations in intervals of the peaks within a predetermined error range. The predetermined error range for the interval of the peaks may be, but is not limited to, ±150 msec. FIG. 17 illustrates an example in which the controller 10 detects the pulse wave having variation in the interval of the peaks within ±150 msec in four consecutive times from the time t₁ to time t₂.

When determining that the stable pulse waves are continuously detected for the predetermined number of times (i.e. Yes in step S201 of FIG. 18), the controller 10 starts the measurement processing of the biological information (step S202). The measurement processing of the biological information is started, for example, at time t₃ in FIG. 17. The controller 10 may store acquired motion factor in the memory 20. When determining that stable pulse waves are continuously detected for the predetermined number of times as described above, the mobile terminal apparatus 1 starts the measurement processing of the biological information.

Next, a predetermined condition for the mobile terminal apparatus 1 to stop the measurement processing of the biological information will be described. In the mobile terminal apparatus 1, the controller 10 may stop the measurement process when a second condition is met. The second condition is a condition for stopping the measurement process. The second condition may be a condition for stopping a part of the measurement process. Here, stopping the measurement process may include, for example, pausing the measurement process. For example, when the second condition is met, the controller 10 may stop at least a part of the measurement process. For example, when the second condition is met, the controller 10 may stop the sleep-inducing function. The stop of the measurement process may be, for example, the end of the application.

The second condition may be a determination, based on the motion factor, that the user has fallen asleep. Autonomously stopping the measurement processing of the biological information when the user has fallen asleep may reduce the power consumption of the mobile terminal apparatus 1 while the user is sleeping. Or, for example, the sleep-inducing function is not necessary after the user falls asleep, thus the mobile terminal apparatus 1 may stop the sleep-inducing function alone. In this case, the mobile terminal apparatus 1 may stop unnecessary functions including the sleep-inducing function while continuing the measurement processing of the biological information such as the respiration rhythm. Thus, the mobile terminal apparatus 1 may reduce its power consumption. For the determination whether the user has fallen asleep, known methods including, for example, the method disclosed in JP-A-2010-273752 may be employed.

The second condition may be determination based on the motion factor that the biological information cannot be measured. For example, when the user falls asleep, the mobile terminal apparatus 1 may be displaced from the measured part or falls to a place where the user is sleeping (e.g., a bed) as the user turns over or the like. In this case, the controller 10 cannot accurately measure the biological information of the user even if continuing the measurement processing of the biological information. Thus, the controller 10 may stop the measurement processing of the biological information. For example, when the controller 10 may determine that the acquired motion factor is different from biological information, the controller 10 may determine that the biological information cannot be measured.

The second condition may be the determination that a motion factor is not detected. For example, when the user falls asleep, the mobile terminal apparatus 1 may be displaced from the measured part or fall to a place where the user is sleeping (e.g., a bed) as the user turns over or the like. In this case, the gyro sensor 12 may not be able to detect the motion factor. When the motion factor is not detected, the mobile terminal apparatus 1 is not pressed against the measured part, and thus the controller 10 cannot measure the biological information of the user. Accordingly, the controller 10 may stop the measurement processing of the biological information.

The second condition may be detection of a change in the positional relationship between the user and the mobile terminal apparatus 1 based on the motion factor. The controller 10 may determine the change in the positional relationship between the user and the mobile terminal apparatus 1 on the basis of output from the acceleration sensor or the gyro sensor 12 of the mobile terminal apparatus 1. That is, when the controller 10 determines that the position of the mobile terminal apparatus 1 is moved on the basis of the output from the acceleration sensor or the gyro sensor 12, the controller 10 may determine that the positional relationship between the user and the mobile terminal apparatus 1 is changed. For example, the user may remove the mobile terminal apparatus 1 from the measured part, or the mobile terminal apparatus 1 may be displaced from the measured part. Thus, the controller 10 may not accurately measure the biological information of the user even if continuing the measurement processing of the biological information. In such cases, accordingly, the controller 10 may stop the measurement processing of the biological information.

The second condition may be detection of a predetermined motion of the user based on the motion factor. The predetermined motion may be any motion that can be detected on the basis of the motion factor. The predetermined motion may be, for example, a motion in which the user changes the respiration rhythm. When the user changes the respiration rhythm, the controller 10 stops the measurement processing of the biological information in accordance with a change in the motion factor caused by the change in the respiration rhythm. For example, the controller 10 may stop the sleep-inducing operation in response to the change in the motion factor caused by the change in the respiration rhythm. Thus, for example, when the user thinks the sleep-inducing operation unnecessary, the user may stop the sleep-inducing operation by changing the respiration rhythm without performing an input operation to the mobile terminal apparatus 1.

The second condition may be detection of a shift of the user to another motion from a measured motion of the biological information based on the motion factor. Another motion may be any motion other than the measured motion of the biological information. For example, when the measured motion is a motion in which the user in a supine position presses the mobile terminal apparatus 1 against the measured part, another motion may include a motion in which the user gets up or turns over. The controller 10 may determine whether the user has shifted to another motion on the basis of the output from the acceleration sensor or the gyro sensor 12 of the mobile terminal apparatus 1. That is, when the controller 10 determines that the position of the mobile terminal apparatus 1 is moved on the basis of the output from the acceleration sensor or the gyro sensor 12, the controller 10 may determine that the user has shifted to another motion. When the user has shifted to another operation, it may be considered that the user is not intending to perform the measurement. Accordingly, the controller 10 may stop the measurement processing of the biological information.

The second condition may be elapse of a predetermined time period after the start of the measurement process. For example, the predetermined time period may be set by the user or automatically set by the mobile terminal apparatus 1. When a predetermined time period has elapsed after the start of the measurement process, the measurement process may not be necessary for reasons such as the user has fallen asleep or a sufficient amount of biological information has already been acquired. As such, when the predetermined time period has elapsed after the start of the measurement process, the controller 10 may stop the measurement processing of the biological information.

The second condition may be determination that the cycle of the motion factor has transitioned to a disturbed state from a stable state. For example, when the mobile terminal apparatus 1 is pressed against a position deviating from the measured part, the cycle of the motion factor becomes unstable and disturbed. In this case, the mobile terminal apparatus 1 may not accurately measure the biological information. As such, when it is determined that the cycle of the motion factor has shifted to a disturbed state from a stable state, the controller 10 may stop the measurement processing of the biological information.

The second condition may be determination that the cycle of the motion factor has transitioned to a stable state from a unstable state. For example, when the respiration rhythm of the user has shifted to a stable state from an unstable state, the cycle of the motion factor has shifted to a stable state from the unstable state. In this case, the user is not having a seizure or the like, and in some cases the biological information does not necessarily need to be recorded. As such, when it is determined that the cycle of the motion factor has shifted from the unstable state to the stable state, the controller 10 may stop the measurement processing of the biological information.

The second condition is not limited to the above examples but may include other conditions. Also, the second condition may include any appropriate combination of the above example conditions and/or other conditions.

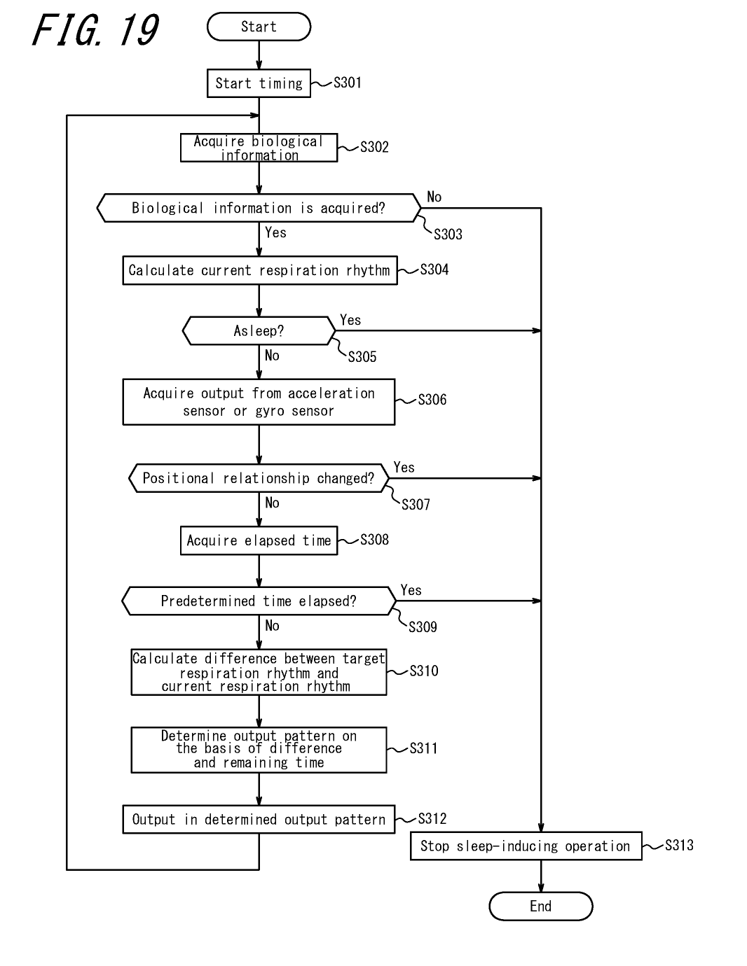

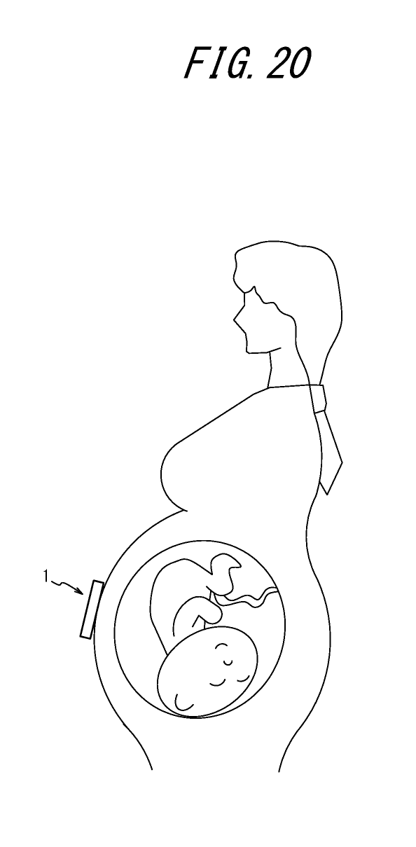

Here, a process to stop the measurement process performed by the controller 10 will be described with reference to FIG. 19. FIG. 19 is a flowchart of the process to stop the measurement processing of the biological information performed by the mobile terminal apparatus 1. Here, it is assumed that the second condition is any of a determination that the motion factor is not detected, a determination that the user has fallen asleep based on the motion factor, a determination of a change in the positional relationship with the user based on the motion factor, and elapse of the predetermined time period after the start of the measurement process. Here, it is also assumed that stopping of the measurement process refers to stopping the sleep-inducing operation serving a part of the measurement process.

The process illustrated in FIG. 19 is performed when, for example, the controller 10 starts the measurement processing of the biological information as a result of the process illustrated in FIG. 18. The target respiration rhythm and an autonomous stop time may be input to the mobile terminal apparatus 1 when, for example, the flow is started. The autonomous stop time is used as a criterion for determining whether the predetermined time period has elapsed as the second condition. The target respiration rhythm and the autonomous stop time may be set by an input operation to the mobile terminal apparatus 1 performed, for example, before the user lies in a supine position.

The mobile terminal apparatus 1 starts timing when the measurement processing of the biological information is started (step S301).

The mobile terminal apparatus 1 operates to acquire the biological information (step S302). The mobile terminal apparatus 1 may acquire the biological information in the manner described in the above embodiment. For example, the mobile terminal apparatus 1 may acquire the information indicating the user's respiration and the information indicating the pulse wave of the user as the biological information.

The mobile terminal apparatus 1 determines whether the biological information is acquired, as the second condition (step S303).

When determining that the biological information is not acquired (i.e. No in step S303), the mobile terminal apparatus 1 stops the sleep-inducing operation (step S313). That is, the mobile terminal apparatus 1 stops the output of the sleep-inducing operation. Then, the mobile terminal apparatus 1 ends the process. In this case, the mobile terminal apparatus 1 may perform, for example, the process for starting the measurement processing of the biological information described with reference to FIG. 18. Or, the mobile terminal apparatus 1 may end the application for measuring the biological information.

When determining that the biological information is acquired (i.e. Yes in step S303), the mobile terminal apparatus 1 calculates the current respiration rhythm (e.g., cycle) of the user (step S304).

Next, the mobile terminal apparatus 1 determines whether the user has fallen asleep on the basis of the acquired biological information, as the second condition (step S305).

When determining that the user has fallen asleep (i.e. Yes in step S305), the mobile terminal apparatus 1 stops the sleep-inducing operation (step S313). Then, the mobile terminal apparatus 1 ends the process.

When determining that the user has not fallen asleep (i.e. No in step S305), the mobile terminal apparatus 1 acquires the output from the acceleration sensor or the gyro sensor 12 (step S306).

The mobile terminal apparatus 1 determines whether the positional relationship between the user and the mobile terminal apparatus 1 is changed on the basis of the output acquired from the acceleration sensor or the gyro sensor 12, as the second condition (step S307).

When determining that the positional relationship between the user and the mobile terminal apparatus 1 is changed (i.e. Yes in step S307), the mobile terminal apparatus 1 stops the sleep-inducing operation (step S313). Then, the mobile terminal apparatus 1 ends the process.

When determining that the positional relationship between the user and the mobile terminal apparatus 1 is not changed (i.e. No in step S307), the mobile terminal apparatus 1 acquires the elapsed time since the start of the timing in step S301 (step S308).

The mobile terminal apparatus 1 determines whether the predetermined time has elapsed, as the second condition (step S309). That is, the mobile terminal apparatus 1 determines whether the elapsed time acquired in step S308 has reached the autonomous stop time that has been set.

When determining that the elapsed time has reached the autonomous stop time (i.e. Yes in step S309), the mobile terminal apparatus 1 stops the sleep-inducing operation (step S313). Then, the mobile terminal apparatus 1 ends the process.

When determining that the elapsed time has not reached the autonomous stop time (i.e. No in step S309), the mobile terminal apparatus 1 calculates the difference between the target respiration rhythm that has been set and the current respiration rhythm calculated in step S304 (step S310).

The mobile terminal apparatus 1 determines the output pattern to be output to the user on the basis of the difference calculated in step S310 and the remaining time to the autonomous stop time (step S311).

The mobile terminal apparatus 1 performs the output in the determined output pattern (step S312). The output may be any manner including, for example, a sound and a vibration that may be recognized by the user. Then, the mobile terminal apparatus 1 proceeds to step S302.

The details of the procedures between step S310 and step S312 may correspond to, for example, the description with reference to FIG. 16.

As described above, the mobile terminal apparatus 1 according to the present embodiment starts or stops the measurement processing of the biological information on the basis of the motion factor. Thus, without the input operation performed by the user to start or stop the measurement processing of the biological information, the mobile terminal apparatus 1 may start or stop the measurement processing of the biological information. Accordingly, the mobile terminal apparatus 1 may reduce the inconvenience that the user may feel in performing the input operation to start or stop the measurement process.

The mobile terminal apparatus 1 may have usage modes other than those described in the above embodiment.

FIG. 20 is a diagram illustrating an another usage mode of the mobile terminal apparatus 1 according to an embodiment. FIG. 20 schematically illustrates a pregnant mother and a fetus. The mobile terminal apparatus 1 according to the above embodiment has been described assuming that the physical information of the user is measured. However, the mobile terminal apparatus 1 according to the present embodiment is not limited to such a usage.

As illustrated in FIG. 20, the user may measure the biological information of the fetus in addition to that of the mother by pressing the mobile terminal apparatus 1 against the abdomen. Generally, a fetus in the early stage of pregnancy (e.g., around 4 to 11 weeks gestation) is so small that it is very difficult to directly listen to the heart sounds of the fetus. At this stage, therefore, ultrasound and so on are often used to check a heart rate of the fetus. However, the mobile terminal apparatus 1 according to the present embodiment is capable of measuring the biological information of the fetus such as by detecting pulsation of the fetus using the gyro sensor 12.

In the usage mode as illustrated in FIG. 20, the mobile terminal apparatus 1 measures the biological information of the fetus together with the biological information of the mother. Thus, the biological information of the fetus may be extracted for use from the biological information measured by the mobile terminal apparatus 1. As described above, the biological information measured by the mobile terminal apparatus 1 may be the biological information of the user's fetus.

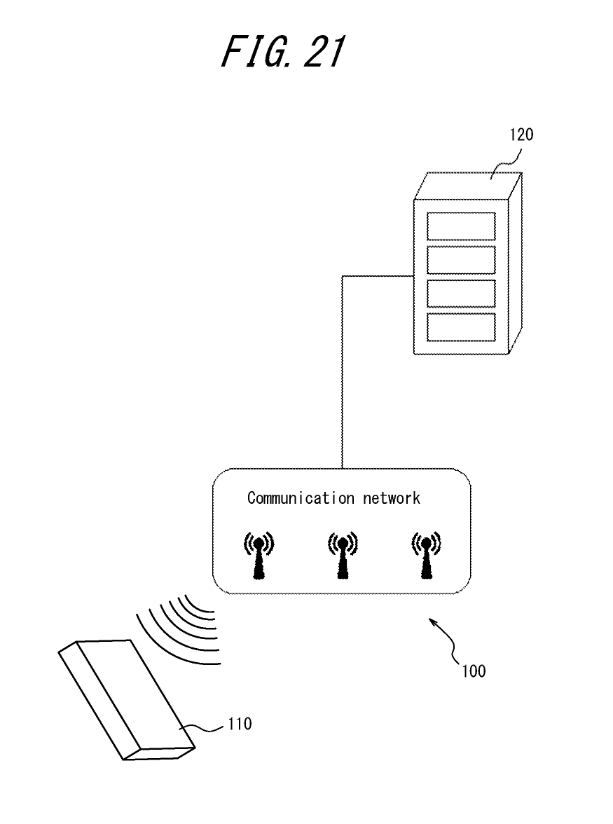

FIG. 21 is a diagram schematically illustrating a biological information measurement system according to an embodiment of the present disclosure. A biological information measurement system 100 according to the embodiment illustrated in FIG. 21 includes a mobile terminal apparatus 110, an external apparatus 120, and communication network.

In the biological information measurement system 100, the mobile terminal apparatus 110 detects the motion factor caused by motion of the user. To that end, the mobile terminal apparatus 110 includes the gyro sensor 12. The mobile terminal apparatus 110 may have the same configuration as the mobile terminal apparatus 1 described above. The mobile terminal apparatus 110 may start and/or stop the measurement processing of the biological information on the basis of the motion factor, as with the mobile terminal apparatus 1 described above. The mobile terminal apparatus 110 includes a communication interface (capable of connecting in a wired or wireless manner) and transmits a detected motion factor to the external apparatus 120. In the biological information measurement system 100, the external apparatus 120 performs various calculations associated with the measurement of the biological information on the basis of a received motion factor. To that end, the external apparatus 120 includes various necessary functional units including the controller 10. Although in FIG. 21 it is assumed that the mobile terminal apparatus 110 and the external apparatus 120 are connected via wireless communication, the biological information measurement system 100 is not limited to such a configuration. For example, the mobile terminal apparatus 110 and the external apparatus 120 may be connected in a wired manner via a predetermined cable or the like. Further, the mobile terminal apparatus 110 may transmit the biological information calculated by the mobile terminal apparatus 110, in place of the detected motion factor, to the external apparatus 120 via the communication interface.

As described above, the biological information measurement system 100 includes the mobile terminal apparatus 110 and the external apparatus 120. The mobile terminal apparatus 110 includes the gyro sensor 12. Here, the gyro sensor 12 detects the motion factor caused by motion of the user's torso while the mobile terminal apparatus 110 is pressed against the user's torso. The external apparatus 120 includes the controller 10. The external apparatus 120 may include an artificial intelligence function, a machine learning function, a deep learning function, etc. and perform various calculations in association with the measurement of the biological information on the basis of the motion factor received from the mobile terminal apparatus 110 by using statistically acquired algorithm.

Several examples have been set forth in order to fully and distinctly describe the present disclosure. However, it should be appreciated that the appended claims should not be construed as being limited to the embodiments described above but should embody all modifications and alternatives that can be created by those who are skilled in the art within the scope of the basic matter described herein. Further, each of the features described in some embodiments may be combined in any manner.

For example, the mobile terminal apparatus 1 and the biological information measurement system 100 according to the present disclosure have been explained. However, embodiments of the present disclosure may be implemented as a biological information measurement method used by the mobile terminal apparatus 1 that includes the gyro sensor 12. In this case, according to the biological information measurement method, the gyro sensor 12 detects the motion factor caused by motion of the user's torso while the mobile terminal apparatus 1 is pressed against the user's torso. Here, the gyro sensor 12 detects the motion factor that is to be processed as a self-control factor. According to the biological information measurement method, also, the measurement processing of the biological information of the user is performed on the basis of the motion factor detected in the state described above.

Further, for example, although the mobile terminal apparatus 1 includes the abutment 40 and the support 50 in the above embodiment, the mobile terminal apparatus 1 may omit the support 50. In this case, a portion on the rear surface of the housing 30 of the mobile terminal apparatus 1 abuts on the user at a position different from the measured part in such a manner that the abutment state of the abutment 40 on the measured part is maintained.

Although in the above embodiment the abutment 40 is fixed to the mobile terminal apparatus 1, the abutment 40 does not necessarily need to be directly fixed to the mobile terminal apparatus 1. The abutment 40 may be fixed to a holder fixed to the mobile terminal apparatus 1.

1. A mobile terminal apparatus comprising:
a gyro sensor configured to detect a motion factor; and
a controller configured to perform measurement processing of biological information of a user on the basis of the motion factor detected due to a movement of the user,
wherein the controller starts or stops the measurement processing on the basis of the motion factor.

2. The mobile terminal apparatus according to claim 1,
wherein the controller is configured to start the measurement processing when a first condition for starting the measurement processing is met.

3. The mobile terminal apparatus according to claim 2,
wherein the first condition is start of the detection of the motion factor.

4. The mobile terminal apparatus according to claim 2,
wherein the first condition is elapse of a predetermined time period from start of detection of the motion factor.

5. The mobile terminal apparatus according to claim 2,
wherein the first condition is continuous detection of the motion factor for a predetermined time.

6. The mobile terminal apparatus according to claim 2,
wherein the first condition is a shift of a cycle of the motion factor to a stable state from a disturbed state.

7. The mobile terminal apparatus according to claim 2,
wherein the first condition is a shift of a cycle of the motion factor to a disturbed state from a stable state.

8. The mobile terminal apparatus according to claim 1,
comprising a communication interface,
wherein the controller is configured to cause the communication interface to transmit at least one of the motion factor and the biological information during the measurement processing.

9. The mobile terminal apparatus according to claim 1,
wherein the controller is configured to stop the measurement processing when a second condition for stopping the measurement processing is met.

10. The mobile terminal apparatus according to claim 9,
wherein the second condition is determination that the user has fallen asleep based on the motion factor.

11. The mobile terminal apparatus according to claim 9,
wherein the second condition is determination that the biological information cannot be measured based on the motion factor.

12. The mobile terminal apparatus according to claim 9,
wherein the second condition is determination that the motion factor is not detected.

13. The mobile terminal apparatus according to claim 9,
wherein the second condition is detection of a change in a positional relationship with the user based on the motion factor.

14. The mobile terminal apparatus according to claim 9,
wherein the second condition is detection of a predetermined motion of the user based on the motion factor.

15. The mobile terminal apparatus according to claim 9,
wherein the second condition is detection of a shift of the user to another motion from a measured motion of the biological information based on the motion factor.

16. The mobile terminal apparatus according to claim 9,
wherein the second condition is elapse of a predetermined time period after start of the measurement operation.

17. The mobile terminal apparatus according to claim 9,
wherein the second condition is determination of a shift of a cycle of the motion factor to a disturbed state from a stable state.

18. The mobile terminal apparatus according to claim 9,
wherein the second condition is determination of a shift of a cycle of the motion factor to a stable state from a disturbed state.

19. A program for causing a mobile terminal apparatus to execute:
a step of detecting a motion factor using a gyro sensor; and
a step of starting or stopping, on the basis of the motion factor, measurement processing of biological information of a user based on the motion factor detected due to a movement of the user.

20. A biological information measurement system comprising:
a mobile terminal apparatus equipped with a gyro sensor configured to detect a motion factor; and
an external apparatus equipped with a controller configured to start or stop, on the basis of the motion factor, measurement processing of biological information of a user based on the motion factor detected due to a movement of the user.
